(19) United States
(12) Patent Application Publication
(10) Pub. No.: US 2025/0075056 A1
(43) Pub. Date: Mar. 6, 2025

(54) IMIDAZOLYSIS OF POLYESTERS AND POLYURETHANES

(71) Applicant: The Board of Trustees of The University of Alabama, Tuscaloosa, AL (US)

(72) Inventors: Jason E. Bara, Tuscaloosa, AL (US); Kathryn Elizabeth O'Harra, Tuscaloosa, AL (US); Mousumi Rani Bepari, Tuscaloosa, AL (US)

(21) Appl. No.: 18/757,750

(22) Filed: Jun. 28, 2024

Related U.S. Application Data
(60) Provisional application No. 63/534,919, filed on Aug. 28, 2023.

(51) Int. Cl. C08J 11/28; C07D 233/58

(52) U.S. Cl. CPC C08J 11/28; C07D 233/58; C08J 2367/02; C08J 2375/04

(57) ABSTRACT

The present disclosure describes processes for the depolymerization of polyesters or polyurethanes and compounds formed by such processes.

### CROSS-REFERENCE TO RELATED APPLICATIONS

This application claims the benefit of priority to U.S. Provisional Application No. 63/534,919 filed Aug. 28, 2023, the disclosure of which is incorporated herein by reference in its entirety.

### STATEMENT REGARDING FEDERALLY SPONSORED RESEARCH OR DEVELOPMENT

This invention was made with government support under Grant No. 2132133 awarded by the National Science Foundation. The Government has certain rights in the invention.

### TECHNICAL FIELD

This disclosure relates to methods of recycling polymers, and more particularly to methods for the lysis and optional subsequent upcycling of polyesters and polyurethanes.

### BACKGROUND

Both polyesters and polyurethanes are commonly used plastics in a variety of products. While in some instances these polymers may be recycled, there is no established technology for the depolymerization of these materials. However, small molecule components recovered from these materials, such as waste plastics and foams, could be used to make new plastics or transformed into different valuable small molecule materials. Thus, there is a clear need for the development of technologies that facilitate the depolymerization of polyesters and/or polyurethanes.

### SUMMARY

The present disclosure provides processes for the depolymerization of polymers such as polyesters or polyurethanes and compounds formed by said processes. The presently disclosed methods do not necessarily require the presence of either solvents or a catalyst to facilitate the depolymerization process.

In one aspect, a process is provided for depolymerizing a polyester comprising treating the polyester with an optionally substituted imidazole to form one or more products comprising one or more optionally substituted 1-acyl imidazole moieties.

In another aspect, a process is provided for depolymerizing a polyurethane comprising treating the polyurethane with an optionally substituted imidazole to form one or more products having one or more optionally substituted 1-carboxamide imidazole moieties.

Compounds formed by the disclosed processes containing one or more optionally substituted 1-acyl imidazole moieties are also provided.

The details of one or more aspects of the disclosure are set forth in the accompanying drawings and the description below. Other features, objects, and advantages of the disclosure will be apparent from the description and drawings, and from the claims.

### DESCRIPTION OF DRAWINGS

FIG. 1 depicts representative schemes of the imidazolysis of polyesters and polyurethanes as described herein.

FIG. 2 depicts further modifications of depolymerization reaction products.

FIG. 3 is a scheme depicting representative steps in the depolymerization of a polyurethane foam.

FIG. 4 is an image depicting isolated polyol products from the depolymerization of a polyurethane foam.

FIGS. 5A-5C depict examples of products formed via aminolysis of PET (5A) with MEA; (5B) with API that can be used in the synthesis of imidazolium ionenes and (5C) expected TBI products of depolymerization of PET by imidazoles.

FIG. 6 depicts the mechanism of small ester imidazolysis by Schmeer (1998).

FIGS. 7A-7B provide FTIR spectra for comparison of depolymerized products with PET (7A) PET with monomers 1b to 4b (7B) PET with monomers 5b to 8b

FIG. 8 depicts ESPs of the evaluated imidazole molecules evaluated at ρ=0.001 e/Bohr³, in which the min and max values (a.u. units) are indicated below the respective molecules in parenthesis.

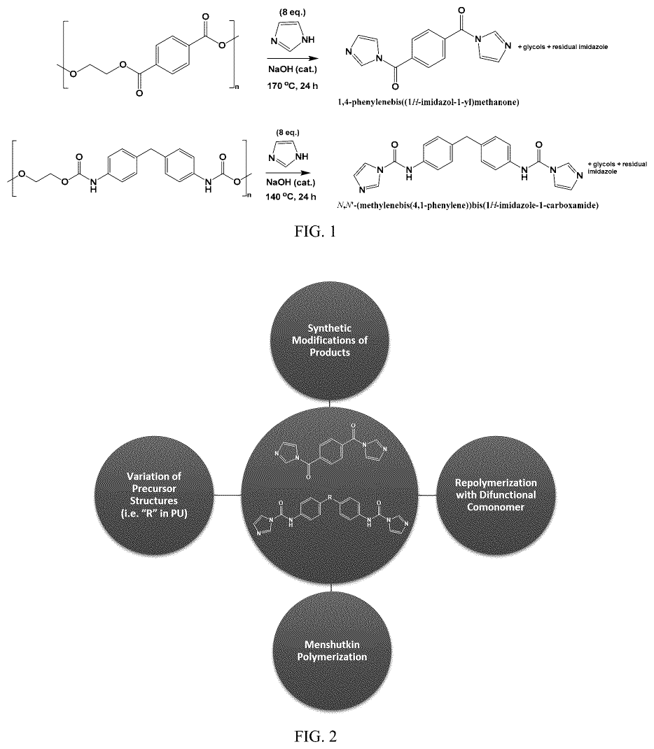

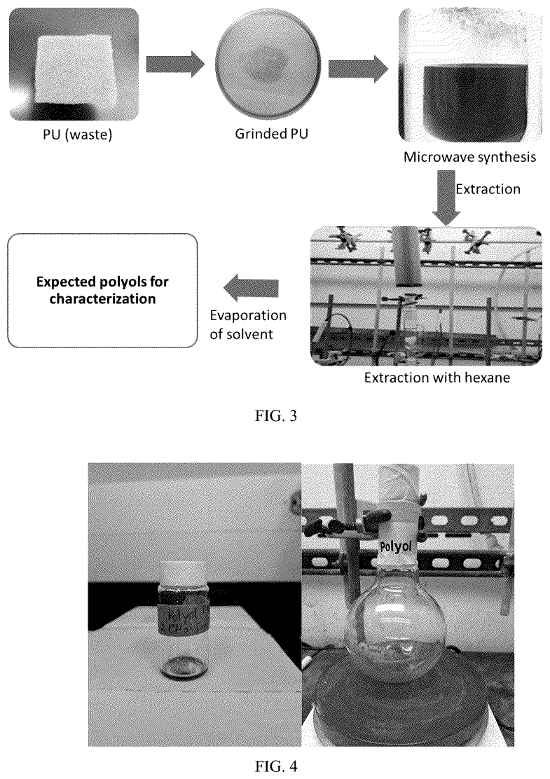

Like reference symbols in the various drawings indicate like elements.

### DETAILED DESCRIPTION

The following description of the disclosure is provided as an enabling teaching of the disclosure in its best, currently known aspects. Many modifications and other aspects disclosed herein will come to mind to one skilled in the art to which the disclosed compositions and methods pertain, benefiting from the teachings presented in the descriptions herein and the associated drawings. Therefore, it is understood that the disclosures are not limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As apparent to those of skill in the art upon reading this disclosure, each of the individual aspects described and illustrated herein has discrete components and features that may be readily separated from or combined with the features of any of the other several aspects without departing from the scope or spirit of the present disclosure.

Any recited method can be carried out in the order of events recited or any other order that is logically possible. Unless otherwise expressly stated, it is in no way intended that any method or aspect set forth herein be construed as requiring that its steps be performed in a specific order. Accordingly, where a method claim does not explicitly state in the claims or descriptions that the steps are to be limited to a particular order, it is in no way intended that an order be inferred in any respect. This holds for any possible non-express basis for interpretation, including logic concerning arrangement of steps or operational flow, meaning derived from grammatical organization or punctuation, or the number or type of aspects described in the specification.

All publications mentioned herein are incorporated by reference to disclose and describe the methods or materials in connection with which the publications are cited. The publications discussed herein are provided solely for their disclosure before the filing date of the present application. Nothing herein is to be construed as an admission that the present invention is not entitled to antedate such publication by prior invention. Further, the dates of publication provided herein can be different from the actual publication dates, which can require independent confirmation.

It is also to be understood that the terminology herein describes particular aspects only and is not intended to be limiting. Unless defined otherwise, all technical and scientific terms herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Before describing the various aspects of the present disclosure, the following definitions are provided and should be used unless otherwise indicated. Additional terms may be defined elsewhere in the present disclosure.

### Definitions

As used herein, “comprising” is interpreted as specifying the presence of the stated features, integers, steps, or components but does not preclude the presence or addition of one or more features, integers, steps, components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, “consisting essentially of” is intended to include examples encompassed by the term “consisting of.”

As used in the specification and the appended claims, the singular forms “a,” “an,” and “the” include plural referents unless the context dictates otherwise. Thus, for example, reference to “a polyurethane,” “a polyester,” or “an imidazole” includes, but is not limited to, two or more such polyurethanes, polyesters, or imidazoles, and the like.

Ratios, concentrations, amounts, and other numerical data can be expressed herein in a range format. Further, the endpoints of each of the ranges are significant both in relation to the other endpoint and independently of the other endpoint. There are many values disclosed herein, and each value is also disclosed as “about” that particular value in addition to the value itself. For example, if the value “10” is disclosed, then “about 10” is also disclosed. Ranges can be expressed herein as from “about” one particular value and to “about” another particular value. Similarly, when values are expressed as approximations, using the antecedent “about,” the particular value forms a further aspect. For example, if the value “about 10” is disclosed, then “10” is also disclosed.

When a range is expressed, a further aspect includes from the one particular value and to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x,’ ‘about y,’ and ‘about z’ as well as the ranges of ‘less than x,’ ‘less than y.’ and ‘less than z.’ Likewise, the phrase ‘about x, y, z, or greater’ should be interpreted to include the specific ranges of ‘about x,’ ‘about y,’ and ‘about z’ as well as the ranges of ‘greater than x,’ greater than y,’ and ‘greater than z.’ In addition, the phrase “about ‘x’ to ‘y’,” where ‘x’ and ‘y’ are numerical values, includes “about ‘x’ to about ‘y’.”

Such a range format is used for convenience and brevity and thus, should be interpreted flexibly to include not only the numerical values explicitly recited as the limits of the range but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, amounts, sizes, formulations, parameters, and other quantities and characteristics are not and need not be exact but may be approximate, larger or smaller, as desired, reflecting tolerances, conversion factors, rounding, measurement error, and the like, and other factors known to those of skill in the art such that equivalent results or effects are obtained. In some circumstances, the value that provides equivalent results or effects cannot be reasonably determined. In such cases, as used herein, “about” and “at or about” mean the nominal value indicated ±10% variation unless otherwise indicated or inferred. In general, an amount, size, formulation, parameter, or other quantity or characteristic is “about,” “approximate,” or “at or about,” whether or not expressly stated to be such. Where “about,” “approximate,” or “at or about” is used before a quantitative value, the parameter also includes the specific quantitative value itself unless expressly stated otherwise.

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates and other isomers, such as rotamers, as if each is specifically described, unless otherwise indicated or otherwise excluded by context. It is to be understood that the compounds provided herein may contain chiral centers. Such chiral centers may be of either the (R-) or (S-) configuration. The compounds provided herein may either be enantiomerically pure, or be diastereomeric or enantiomeric mixtures. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Compounds described herein may contain one or more double bonds and, thus, potentially give rise to cis/trans (E/Z) isomers, as well as other conformational isomers. Unless states to the contrary, all such possible isomers are contemplated, as well as mixtures of such isomers.

Compounds described herein may also present as an equilibrium of tautomers. For example, ketones with an α-hydrogen can exist in an equilibrium of the keto form and the enol form. Likewise, amides with an N-hydrogen can exist in an equilibrium of the amide form and the imidic acid form. Unless stated to the contrary, all possible tautomers of the compounds described herein are contemplated.

A dash (“-”) that is not between two letters or symbols is used to indicate a point of attachment for a substituent. For example, —(C═O)NH₂ is attached through the carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the invention and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

The terms for various functional groups as used herein are not intended to be limited to monovalent radicals and may include polyvalent radical groups as appropriate, such as divalent, trivalent, tetravalent, pentavalent, and hexavalent groups, and the like, based on the position and location of such groups in the compounds described herein as would be readily understood by the skilled person in the context in which said functional groups are recited.

As used herein, the symbol

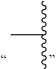

(which hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

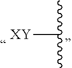

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

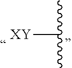

infers that when R³ is “XY”, the point of attachment bond is the same bond as the bond by which R³ is depicted as being bonded to CH₃.

“Alkyl” is a straight chain or branched saturated aliphatic hydrocarbon group. In certain aspects, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₆alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙalkyl is used herein in conjunction with another group, for example (C₃-C₇cycloalkyl)C₀-C₄alkyl, or —C₀-C₄(C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In some aspects, the alkyl group is optionally substituted as described herein. The term “alkyl” as used herein is not intended to be limited to monovalent radicals and may include polyvalent radical groups as appropriate, such as divalent, trivalent, tetravalent, pentavalent, and hexavalent alkyl, and the like, based on the position and location of such groups in the compounds described herein as would be readily understood by the skilled person.

“Cycloalkyl” is a saturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In some aspects, the cycloalkyl group is optionally substituted as described herein. The term “cycloalkyl” as used herein is not intended to be limited to monovalent radicals and may include polyvalent radical groups as appropriate, such as divalent, trivalent, tetravalent, pentavalent, and hexavalent cycloalkyl, and the like, based on the position and location of such groups in the compounds described herein as would be readily understood by the skilled person.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one aspect, the alkenyl group is optionally substituted as described herein. The term “alkenyl” as used herein is not intended to be limited to monovalent radicals and may include polyvalent radical groups as appropriate, such as divalent, trivalent, tetravalent, pentavalent, and hexavalent alkenyl, and the like, based on the position and location of such groups in the compounds described herein as would be readily understood by the skilled person.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one aspect, the alkynyl group is optionally substituted as described herein. The term “alkynyl” as used herein is not intended to be limited to monovalent radicals and may include polyvalent radical groups as appropriate, such as divalent, trivalent, tetravalent, pentavalent, and hexavalent alkynyl, and the like, based on the position and location of such groups in the compounds described herein as would be readily understood by the skilled person.

“Alkoxy” is an alkyl group as defined above covalently bound through an oxygen bridge (—O—). Examples of alkoxy include, but are not limited to, methoxy, ethoxy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group as defined above with the indicated number of carbon atoms covalently bound through a sulfur bridge (—S—).

“Alkanoyl” is an alkyl group as defined above covalently bound through a carbonyl (C═O) bridge. The carbonyl carbon is included in the number of carbons, for example C₂alkanoyl is a CH₃(C═O)— group. In one aspect, the alkanoyl group is optionally substituted as described herein.

“Halo” or “halogen” indicates, independently, any of fluoro, chloro, bromo or iodo.

“Aryl” indicates an aromatic group containing only carbon in the aromatic ring or rings. In one aspect, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4- to 7- or 5- to 7-membered saturated or partially unsaturated cyclic group that optionally contains 1, 2, or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one aspect, aryl groups are pendant. An example of a pendant ring is a phenyl group substituted with a phenyl group. In one aspect, the aryl group is optionally substituted as described herein. The term “aryl” as used herein is not intended to be limited to monovalent radicals and may include polyvalent radical groups as appropriate, such as divalent, trivalent, tetravalent, pentavalent, and hexavalent aryl, and the like, based on the position and location of such groups in the compounds described herein as would be readily understood by the skilled person.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S— portions. Examples of saturated heterocycle groups including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but are not limited, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3,-dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include, but are not limited to, partially unsaturated condensed heterocyclic groups containing 1 to 5 nitrogen atoms, for example indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms. The term “heterocycle” as used herein is not intended to be limited to monovalent radicals and may include polyvalent radical groups as appropriate, such as divalent, trivalent, tetravalent, pentavalent, and hexavalent heterocycle, and the like, based on the position and location of such groups in the compounds described herein as would be readily understood by the skilled person.

“Heteroaryl” refers to a stable monocyclic, bicyclic, or multicyclic aromatic ring which contains from 1 to 4, or in some aspects 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 4, or in some aspects from 1 to 3 or from 1 to 2, heteroatoms selected from N, O, S, B, or P, with remaining ring atoms being carbon. In one aspects, the only heteroatom is nitrogen. In one aspect, the only heteroatom is oxygen. In one aspect, the only heteroatom is sulfur. Monocyclic heteroaryl groups typically have from 5 to 6 ring atoms. In some aspects, bicyclic heteroaryl groups are 8- to 10-membered heteroaryl groups, that is groups containing 8 or 10 ring atoms in which one 5-, 6-, or 7-membered aromatic ring which contains from 1 to 4 heteroatoms selected from N, O, S, B, or P is fused to a second aromatic or non-aromatic ring, wherein the point of attachment is an aromatic ring. When the total number of S and O atoms in the heteroaryl ring exceeds 1, these heteroatoms are not adjacent to one another within the ring. In one aspect, the total number of S and O atoms in the heteroaryl ring is not more than 2. In another aspect, the total number of S and O atoms in the heteroaryl ring is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The term “heteroaryl” as used herein is not intended to be limited to monovalent radicals and may include polyvalent radical groups as appropriate, such as divalent, trivalent, tetravalent, pentavalent, and hexavalent heteroaryl, and the like, based on the position and location of such groups in the compounds described herein as would be readily understood by the skilled person.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers, such as Sigma-Aldrich (formally MilliporeSigma, Burlington, MA) or Thermo Fisher Scientific Inc. (Waltham, MA), or are prepared by methods known to those skilled in the art following procedures set forth in references such as Fieser and Fieser's Reagents for Organic Synthesis (John Wiley and Sons, 2007); Organic Reactions (John Wiley and Sons, 2004); March's Advanced Organic Chemistry, (John Wiley and Sons, 8^{th} Edition); and Larock's Comprehensive Organic Transformations (John Wiley and Sons, 3^{rd} edition, 2017).

### Imidazolysis of Polyesters

In one aspect, a process is provided for depolymerizing a polyester. In some aspects, the process comprises treating the polyester with an optionally substituted imidazole. In some aspects, the process forms one or more products comprising one or more optionally substituted 1-acyl imidazole moieties.

Any suitable polyester may be used in the processes described herein, such as a polyester formed by condensation of a dicarboxylic acid and a diol or a polyester formed by condensation of a hydroxy-substituted acid or ester or derivative thereof, such as a lactone

In some aspects, the polyester was formed by condensation of a dicarboxylic acid and a diol. In some aspects the polyester is selected from the group consisting of poly(ethylene terephthalate) (PET), poly(trimethylene terephthalate) (PTT), poly(butylene terephthalate) (PBT), poly(ethylene isophthalate), poly(octamethylene terephthalate), poly(decamethylene terephthalate), poly(pentamethylene isophthalate), poly(butylene isophthalate), poly(hexamethylene isophthalate), poly(hexamethylene adipate), poly(pentamethylene adipate), poly(pentamethylene sebacate), poly(hexamethylene sebacate), poly(1,4-cyclohexylene terephthalate), poly(1,4-cyclohexylene sebacate), poly(ethylene terephthalate-co-sebacate), and poly(ethylene-co-tetramethylene terephthalate).

In some aspects, the one or more products comprise one or more 1-acyl imidazole derivatives of terephthalic acid, isophthalic acid, 2,6-naphthalene dicarboxylic acid, 4,4′-diphenylether dicarboxylic acid, hexahydrophthalic acid, 2,7-naphthalenedicarboxylic acid, phthalic acid, 4,4′-methylenebis(benzoic acid), oxalic acid, malonic acid, succinic acid, methyl succinic acid, glutaric acid, adipic acid, 3-methyladipic acid, pimelic acid, suberic acid, azelaic acid, sebacic acid, 1,11-undecanedicarboxylic acid, 1,10-decanedicarboxylic acid, hexadecanedioic acid, docosanedioic acid, tetracosanedioic acid, 1,4-cyclohexanedicarboxylic acid, 1,3-cyclohexanedicarboxylic acid, 1,2-cyclohexanediacetic acid, fumaric acid, maleic acid, or combinations thereof.

In some aspects, the one or more products comprise a compound selected from:

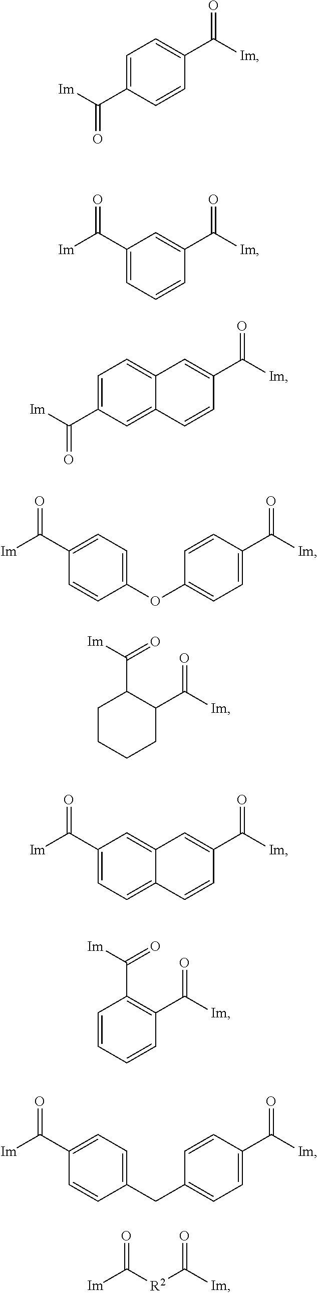

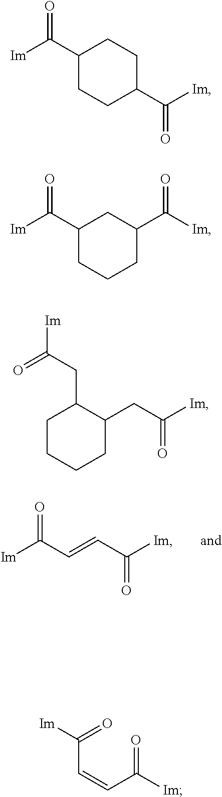

wherein Im is independently selected at each occurrence from:

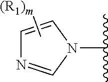

wherein
m is 0, 1, 2, or 3;
R₁ is independently selected at each occurrence from hydrogen, halo, cyano, azido, nitro, silyl, hydroxy, amino, aldehyde, carboxylic acid, ester, C₁-C₆ alkyl, C₁-C₆ haloalkyl, C₂-C₆ alkenyl, C₂-C₆ alkynyl, C₁-C₆ alkoxy, C₃-C₆ cycloalkyl, 3- to 8-membered monocyclic or bicyclic heterocycle, 6- to 10-membered monocyclic or bicyclic aryl, 5- to 10-membered monocyclic or bicyclic heteroaryl, sulfo-oxo, sulfonyl, sulfone, sulfoxide, sulfonylamino, and thiol; and
R² is selected from C₀-C₂₂ alkyl which may be optionally substituted with one or more C₁-C₆ alkyl groups.

In some aspects, the one or more products further comprise one or more polyols. Representative examples of such polyols include, but are not limited to, ethylene glycol, propylene glycol, octamethylene glycol, decamethylene glycol, pentamethylene glycol, butylene glycol, hexamethylene glycol, and 1,4-cyclohexylene diol.

In some aspects, the polyester was formed by condensation of a hydroxy-substituted acid or ester or derivative thereof, such as a lactone. In some aspects, the polyester is selected from polyglycolic acid (PGA), polylactic acid (PLA), polycaprolactone (PCL), polyhydroxybutyrate (PHB), or a polyhydroxyalkanoate (PHA).

In some aspects, the one or more products comprise one or more 1-acyl imidazole derivatives of glycolic acid, lactic acid, hydroxycaproic acid, hydroxybutyric acid, or a hydroxyalkanoic acid, or combinations thereof. In some aspects, the one or more products comprise a compound selected from:

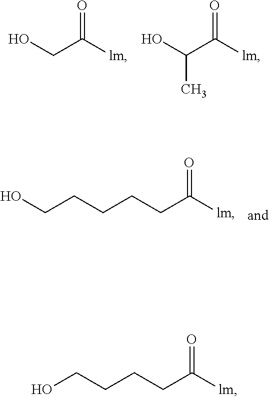

wherein all variables are as defined herein.

In one aspect, the polyester comprises polyethylene terephthalate (PET).

Any suitable optionally substituted imidazole may be used in the processes described herein. In some aspects, the optionally substituted imidazole comprises a compound of Formula I

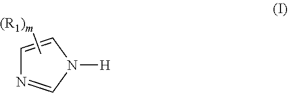

wherein all variables are as defined herein.

In some aspects, the optionally substituted imidazole is selected from imidazole, 2-ethylimidazole, 2,4,5-trimethylimidazole, 2-isopropylimidazole, and 4-methylimidazole.

In some aspects, the optionally substituted imidazole is present in an amount of about 2 molar equivalents to about 10 molar equivalents relative to the polyester, including exemplary values of about 2 molar equivalents, of about 3 molar equivalents, of about 4 molar equivalents, of about 5 molar equivalents, of about 6 molar equivalents, of about 7 molar equivalents, of about 8 molar equivalents, of about 9 molar equivalents, of about 10 molar equivalents, a range formed from any of the foregoing values, or a sub-range within any such range.

In some aspects, the process is performed in the presence of a base. In some aspects, the base comprises a hydroxide or a carbonate. In some aspects, the base comprises a hydroxide, for example, an alkali metal or alkaline earth metal hydroxide. Representative examples of hydroxides which may be used include, but are not limited to, lithium hydroxide, sodium hydroxide, potassium hydroxide, rubidium hydroxide, cesium hydroxide, magnesium hydroxide, calcium hydroxide, strontium hydroxide, and barium hydroxide. In some aspects, the base comprises a carbonate, for example lithium carbonate, sodium carbonate, potassium carbonate, rubidium carbonate, cesium carbonate, and ammonium carbonate.

In some aspects, the base is present in an amount of about 0.05 molar equivalents to about 0.6 molar equivalents relative to the polyester, including exemplary values of about 0.05 molar equivalents, about 0.1 molar equivalents, about 0.15 molar equivalents, about 0.2 molar equivalents, about 0.25 molar equivalents, about 0.3 molar equivalents, about 0.35 molar equivalents, about 0.4 molar equivalents, about 0.45 molar equivalents, about 0.5 molar equivalents, about 0.55 molar equivalents, about 0.6 molar equivalents, a range formed from any of the foregoing values, or a sub-range within any such range.

In some aspects, the process is performed at a temperature from about 100 degrees Celsius to about 200 degrees Celsius, including exemplary values of about 100 degrees Celsius, about 110 degrees Celsius, about 120 degrees Celsius, about 130 degrees Celsius, about 140 degrees Celsius, about 150 degrees Celsius, about 160 degrees Celsius, about 170 degrees Celsius, about 180 degrees Celsius, about 190 degrees Celsius, about 200 degrees Celsius, a range formed from any of the foregoing values, or a sub-range within any such range. In some aspects, the process is performed at a temperature greater than the melting point of the polyester to be depolymerized.

In some aspects, the process may be performed by the use of microwave irradiation. Microwaves act as high frequency electric fields and will generally heat any material containing mobile electric charges. Component molecules are forced to rotate with the field and lose energy in collisions. Acting as an internal heat source, microwave absorption is able to heat the target compounds without heating the entire furnace or oil bath, which saves time and energy. Numerous microwave reactors in which the above processes can be performed are commercially available and would be familiar to a person of ordinary skill in the art.

The processes described herein may be performed in any suitable solvent or in the absence of a solvent. In typical aspects, the process is performed in the absence of a solvent.

In another aspect, a compound is provided comprising one or more optionally substituted 1-acyl imidazole moieties formed by the processes described herein.

### Imidazolysis of Polyurethanes

In another aspect, a process is provided for depolymerizing a polyurethane. In some aspects, the process comprises treating the polyurethane with an optionally substituted imidazole. In some aspects, the process form one or more products having one or more optionally substituted 1-carboxamide imidazole moieties.

Any suitable polyurethane may be used in the disclosed processes. In some aspects, the polyurethane was formed by condensation of one or more diisocyanates, one or more polyols, optionally one or more chain extenders, and optionally one or more crosslinking agents.

In some aspects, the one or more diisocyanates are selected from an aliphatic diisocyanate, an aromatic diisocyanate, or combinations thereof. In some aspects, the one or more diisocyanates are selected from 4,4′-dicyclohexylmethane diisocyanate, isophorone diisocyanate, 1,4-cyclohexylmethane diisocyanate, 4,4′-diphenylmethane diisocyanate, 2,4-toluene diisocyanate, 2,6-toluene diisocyanate, hexamethylene diisocyanate, or combinations thereof.

In some aspects, the one or more polyols are selected from a polyether polyol, a polycarbonate polyol, a polyester polyol, a silicone polyol, or combinations thereof. In some aspects, the one or more polyols are selected from polyethylene glycol, polypropylene glycol, polytetramethyleneglycol, poly(1,6-hexamethylene carbonate)diol, poly(decamethylene carbonate)diol, oligocarbonate diol, poly(hexamethylene-pentamethylene carbonate)diol, polylactide, polycaprolactone, polyethylene glycol adipate, polydimethylsiloxane, polyaryl siloxane, and polyalkyl siloxane.

In some aspects, the one or more products comprise one or more 1-carboxamide imidazole derivatives of 4,4′-dicyclohexylmethane diisocyanate, isophorone diisocyanate, 4,4′-diphenylmethane diisocyanate, 2,4-toluene diisocyanate, 2,6-toluene diisocyanate, hexamethylene diisocyanate, or combinations thereof. In some aspects, the one or more products comprise a compound selected from:

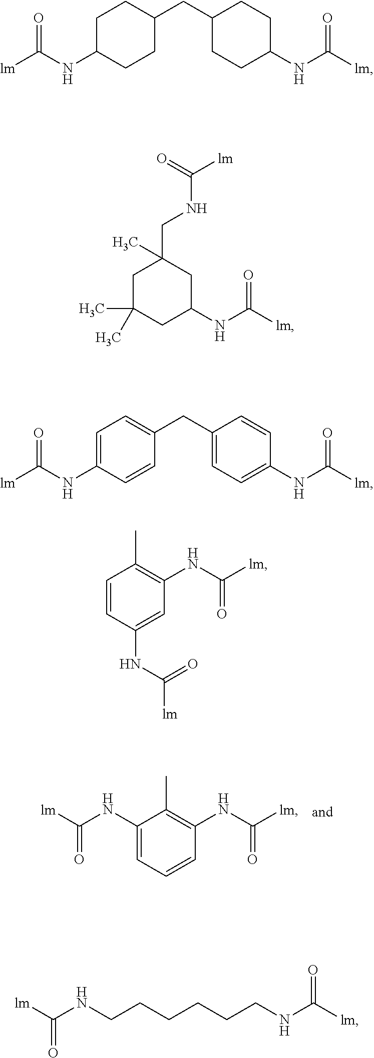

wherein all variables are as defined herein.

In some aspects, the one or more products further comprise one or more polyols. Representative examples of such polyols include, but are not limited to, polyether polyols, polycarbonate polyols, polyester polyols, silicone polyols, polyethylene glycol, polypropylene glycol, polytetramethyleneglycol, poly(1,6-hexamethylene carbonate)diol, poly(decamethylene carbonate)diol, oligocarbonate diol, poly(hexamethylene-pentamethylene carbonate)diol, polylactide, polycaprolactone, polyethylene glycol adipate, polydimethylsiloxane, polyaryl siloxane, and polyalkyl siloxane.

Any suitable optionally substituted imidazole may be used in the processes described herein. In some aspects, the optionally substituted imidazole comprises a compound of Formula I

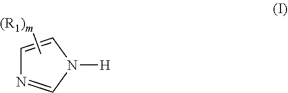

wherein all variables are as defined herein.

In some aspects, the optionally substituted imidazole is selected from imidazole, 2-ethylimidazole, 2,4,5-trimethylimidazole, 2-isopropylimidazole, and 4-methylimidazole.

In some aspects, the optionally substituted imidazole is present in an amount of about 2 molar equivalents to about 10 molar equivalents relative to the polyester, including exemplary values of about 2 molar equivalents, of about 3 molar equivalents, of about 4 molar equivalents, of about 5 molar equivalents, of about 6 molar equivalents, of about 7 molar equivalents, of about 8 molar equivalents, of about 9 molar equivalents, of about 10 molar equivalents, a range formed from any of the foregoing values, or a sub-range within any such range.

In some aspects, the process is performed in the presence of a base. In some aspects, the base comprises a hydroxide or a carbonate. In some aspects, the base comprises a hydroxide, for example, an alkali metal or alkaline earth metal hydroxide. Representative examples of hydroxides which may be used include, but are not limited to, lithium hydroxide, sodium hydroxide, potassium hydroxide, rubidium hydroxide, cesium hydroxide, magnesium hydroxide, calcium hydroxide, strontium hydroxide, and barium hydroxide. In some aspects, the base comprises a carbonate, for example lithium carbonate, sodium carbonate, potassium carbonate, rubidium carbonate, cesium carbonate, and ammonium carbonate.

In some aspects, the base is present in an amount of about 0.05 molar equivalents to about 0.6 molar equivalents relative to the polyester, including exemplary values of about 0.05 molar equivalents, about 0.1 molar equivalents, about 0.15 molar equivalents, about 0.2 molar equivalents, about 0.25 molar equivalents, about 0.3 molar equivalents, about 0.35 molar equivalents, about 0.4 molar equivalents, about 0.45 molar equivalents, about 0.5 molar equivalents, about 0.55 molar equivalents, about 0.6 molar equivalents, a range formed from any of the foregoing values, or a sub-range within any such range.

In some aspects, the process is performed at a temperature from about 100 degrees Celsius to about 200 degrees Celsius, including exemplary values of about 100 degrees Celsius, about 110 degrees Celsius, about 120 degrees Celsius, about 130 degrees Celsius, about 140 degrees Celsius, about 150 degrees Celsius, about 160 degrees Celsius, about 170 degrees Celsius, about 180 degrees Celsius, about 190 degrees Celsius, about 200 degrees Celsius, a range formed from any of the foregoing values, or a sub-range within any such range. In some aspects, the process is performed at a temperature greater than the melting point of the polyurethane to be depolymerized.

In some aspects, the process may be performed by the use of microwave irradiation. Microwaves act as high frequency electric fields and will generally heat any material containing mobile electric charges. Component molecules are forced to rotate with the field and lose energy in collisions. Acting as an internal heat source, microwave absorption is able to heat the target compounds without heating the entire furnace or oil bath, which saves time and energy. Numerous microwave reactors in which the above processes can be performed are commercially available and would be familiar to a person of ordinary skill in the art.

The processes described herein may be performed in any suitable solvent or in the absence of a solvent. In typical aspects, the process is performed in the absence of a solvent.

In another aspect, a compound is provided comprising one or more optionally substituted 1-carboxamide imidazole moieties formed by the processes described herein.

Variations on compounds used in the processes for the preparation of compounds of Formula I can include the addition, subtraction, or movement of various constituents as described for each compounds. Similarly, when one or more chiral centers is present in a molecule, the chirality of the molecule can be changed. Additionally, the synthesis of the compounds used in these processes can involve the protection of various chemical groups, and further the compounds prepared by the disclosed processes may be subsequently deprotected as appropriate. The use of protection and deprotection, and the selection of appropriate protecting groups, would be readily known to one skilled in the art. “Protecting group”, as used herein, refers to any convention functional group that allows one to obtain chemoselectivity in a subsequent chemical reaction. Protecting groups are described, for example, in Peter G. M. Wuts, Greene's Protective Groups in Organic Synthesis, 5^{th} Ed., Wiley & Sons, 2014. For a particular compound and/or a particular chemical reaction, a person skilled in the art knows how to select and implement appropriate protecting groups and their associated synthetic methods. Examples of amine protecting groups include acyl and alkoxy carbonyl groups, such a t-butoxycarbonyl (BOC) and [2-(trimethylsilyl)ethoxy]methoxy (SEM). Examples of carboxyl protecting groups include C₁-C₆ alkoxy groups, such as methyl, ethyl, and t-butyl. Examples of alcohol protecting groups include benzyl, trityl, silyl ethers, and the like.

The described processes, or reaction to produce the compounds used in the described processes, can be carried out in solvents indicated herein, or in solvents which can be selected by one of skill in the art of organic synthesis. Solvents can be substantially nonreactive with the starting materials (reactants), intermediates, or products under the conditions at which the reaction is carried out, i.e., temperature and pressure. Reactions can be carried out in one solvent or a mixture of more than one solvent. In preferred aspects, the reactions described herein are carried out in the absence of solvent. Product or intermediate formation can be monitored according to any suitable method known in the art. For example, product formation can be monitored by spectroscopic means, such as nuclear magnetic resonance spectroscopy (e.g., ¹H and ¹³C), infrared spectroscopy, spectrophotometry (e.g., UV-visible), or mass spectrometry, or by chromatography such as high performance liquid chromatography (HPLC) or thin layer chromatography (TLC).

In view of the described processes and compositions, hereinbelow are described certain more particular aspects of the disclosure. These particularly recited aspects should not, however, be interpreted to have any limiting effect on any different claims containing different or more general teachings described herein, or that the “particular” aspects are somehow limited in some way other than the inherent meanings of the language and formulae literally used therein.

### Additional Aspects

Aspect A1. A process for depolymerizing a polyester comprising treating the polyester with an optionally substituted imidazole in the presence of a base to form one or more products comprising one or more optionally substituted 1-acyl imidazole moieties.

Aspect A2. The process of aspect A1, wherein the polyester was formed by condensation of a dicarboxylic acid and a polyol.

Aspect A3. The process of aspect A1 or A2, wherein the polyester is selected from poly(ethylene terephthalate) (PET), poly(trimethylene terephthalate) (PTT), poly(butylene terephthalate) (PBT), poly(ethylene isophthalate), poly(octamethylene terephthalate), poly(decamethylene terephthalate), poly(pentamethylene isophthalate), poly(butylene isophthalate), poly(hexamethylene isophthalate), poly(hexamethylene adipate), poly(pentamethylene adipate), poly(pentamethylene sebacate), poly(hexamethylene sebacate), poly(1,4-cyclohexylene terephthalate), poly(1,4-cyclohexylene sebacate), poly(ethylene terephthalate-co-sebacate), and poly(ethylene-co-tetramethylene terephthalate).

Aspect A4. The process of any one of aspects A1-A3, wherein the one or more products comprise one or more 1-acyl imidazole derivatives of terephthalic acid, isophthalic acid, 2,6-naphthalene dicarboxylic acid, 4,4′-diphenylether dicarboxylic acid, hexahydrophthalic acid, 2,7-naphthalenedicarboxylic acid, phthalic acid, 4,4′-methylenebis(benzoic acid), oxalic acid, malonic acid, succinic acid, methyl succinic acid, glutaric acid, adipic acid, 3-methyladipic acid, pimelic acid, suberic acid, azelaic acid, sebacic acid, 1,11-undecanedicarboxylic acid, 1,10-decanedicarboxylic acid, hexadecanedioic acid, docosanedioic acid, tetracosanedioic acid, 1,4-cyclohexanedicarboxylic acid, 1,3-cyclohexanedicarboxylic acid, 1,2-cyclohexanediacetic acid, fumaric acid, maleic acid, or combinations thereof.

Aspect A5. The process of any one of aspects A1-A4, wherein the one or more products further comprise one or more polyols.

Aspect A6. The process of aspect A1, wherein the polyester was formed by condensation of a hydroxy-substituted acid or ester or derivative thereof, such as a lactone.

Aspect A7. The process of aspect A1 or A6, wherein the polyester is selected from polyglycolic acid (PGA), polylactic acid (PLA), polycaprolactone (PCL), polyhydroxybutyrate (PHB), or a polyhydroxyalkanoate (PHA).

Aspect A8. The process of any one of aspects A1 or A6-A7, wherein the one or more products comprise one or more 1-acyl imidazole derivatives of glycolic acid, lactic acid, hydroxycaproic acid, hydroxybutyric acid, or a hydroxyalkanoic acid, or combinations thereof.

Aspect A9. The process of aspect A1, wherein the polyester comprises polyethylene terephthalate (PET).

Aspect A10. The process of any one of aspects A1-A9, wherein the optionally substituted imidazole comprises a compound of Formula I

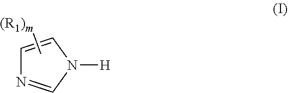

wherein
m is 0, 1, 2, or 3; and
R₁ is independently selected at each occurrence from hydrogen, halo, cyano, azido, nitro, silyl, hydroxy, amino, aldehyde, carboxylic acid, ester, C₁-C₆ alkyl, C₁-C₆ haloalkyl, C₂-C₆ alkenyl, C₂-C₆ alkynyl, C₁-C₆ alkoxy, C₃-C₆ cycloalkyl, 3- to 8-membered monocyclic or bicyclic heterocycle, 6- to 10-membered monocyclic or bicyclic aryl, 5- to 10-membered monocyclic or bicyclic heteroaryl, sulfo-oxo, sulfonyl, sulfone, sulfoxide, sulfonylamino, and thiol.

Aspect A11. The process of any one of aspects A1-A10, wherein the optionally substituted imidazole is selected from imidazole, 2-ethylimidazole, 2,4,5-trimethylimidazole, 2-isopropylimidazole, and 4-methylimidazole.

Aspect A12. The process of any one of aspects A1-A11, wherein the optionally substituted imidazole is present in an amount of about 2 molar equivalents to about 10 molar equivalents relative to the polyester.

Aspect A13. The process of any one of aspects A1-A12, wherein the base comprises a hydroxide or a carbonate.

Aspect A14. The process of any one of aspects A1-A13, wherein the base comprises sodium hydroxide or potassium hydroxide.

Aspect A15. The process of any one of aspects A1-A14, wherein the base is present in an amount of about 0.05 molar equivalents to about 0.6 molar equivalents relative to the polyester.

Aspect A16. The process of any one of aspects A1-A15, wherein the process is performed at a temperature from about 100 degrees Celsius to about 200 degrees Celsius.

Aspect A17. The process of any one of aspects A1-A16, wherein the process is performed in the absence of a solvent.

Aspect A18. A compound comprising one or more optionally substituted 1-acyl imidazole moieties formed by the process of any one of aspects A1-A17.

Aspect A19. A process for depolymerizing a polyurethane comprising treating the polyurethane with an optionally substituted imidazole in the presence of a base to form one or more products having one or more optionally substituted 1-carboxamide imidazole moieties.

Aspect A20. The process of aspect A19, wherein the polyurethane was formed by condensation of one or more diisocyanates, one or more polyols, optionally one or more chain extenders, and optionally one or more crosslinking agents.

Aspect A21. The process of aspect A20, wherein the one or more diisocyanates are selected from an aliphatic diisocyanate, an aromatic diisocyanate, or combinations thereof.

Aspect A22. The process of aspect A21, wherein the one or more diisocyanates are selected from 4,4′-dicyclohexylmethane diisocyanate, isophorone diisocyanate, 4,4′-diphenylmethane diisocyanate, 2,4-toluene diisocyanate, 2,6-toluene diisocyanate, hexamethylene diisocyanate, or combinations thereof.

Aspect A23. The process of aspect A20, wherein the one or more polyols are selected from a polyether polyol, a polycarbonate polyol, a polyester polyol, a silicone polyol, or combinations thereof.

Aspect A24. The process of aspect A23, wherein the one or more polyols are selected from polyethylene glycol, polypropylene glycol, polytetramethyleneglycol, poly(1,6-hexamethylene carbonate)diol, poly(decamethylene carbonate)diol, oligocarbonate diol, poly(hexamethylene-pentamethylene carbonate)diol, polylactide, polycaprolactone, polyethylene glycol adipate, polydimethylsiloxane, polyaryl siloxane, and polyalkyl siloxane.

Aspect A25. The process of any one of aspects A19-A24, wherein one or more products comprise one or more 1-carboxamide imidazole derivatives of 4,4′-dicyclohexylmethane diisocyanate, isophorone diisocyanate, 1,4-cyclohexylmethane diisocyanate, 4,4′-diphenylmethane diisocyanate, 2,4-toluene diisocyanate, 2,6-toluene diisocyanate, hexamethylene diisocyanate, or combinations thereof.

Aspect A26. The process of any one of aspects A19-A25, wherein the one or more products further comprises one or more polyols.

Aspect A27. The process of any one of aspects A19-A26, wherein the optionally substituted imidazole comprises a compound of Formula I

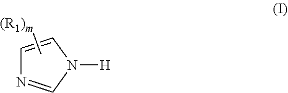

wherein
m is 0, 1, 2, or 3; and
R₁ is independently selected at each occurrence from hydrogen, halo, cyano, azido, nitro, silyl, hydroxy, amino, aldehyde, carboxylic acid, ester, C₁-C₆ alkyl, C₁-C₆ haloalkyl, C₂-C₆ alkenyl, C₂-C₆ alkynyl, C₁-C₆ alkoxy, C₃-C₆ cycloalkyl, 3- to 8-membered monocyclic or bicyclic heterocycle, 6- to 10-membered monocyclic or bicyclic aryl, 5- to 10-membered monocyclic or bicyclic heteroaryl, sulfo-oxo, sulfonyl, sulfone, sulfoxide, sulfonylamino, and thiol.

Aspect A28. The process of any one of aspects A19-A27, wherein the optionally substituted imidazole is selected from imidazole, 2-ethylimidazole, 2,4,5-trimethylimidazole, 2-isopropylimidazole, and 4-methylimidazole.

Aspect A29. The process of any one of aspects A19-A28, wherein the optionally substituted imidazole is present in an amount of about 2 molar equivalents to about 10 molar equivalents relative to the polyurethane.

Aspect A30. The process of any one of aspects A19-A29, wherein the base comprises a hydroxide or a carbonate.

Aspect A31. The process of any one of aspects A19-A20, wherein the base comprises sodium hydroxide or potassium hydroxide.

Aspect A32. The process of any one of aspects A19-A31, wherein the base is present in an amount of about 0.05 molar equivalents to about 0.6 molar equivalents relative to the polyurethane.

Aspect A33. The process of any one of aspects A19-A32, wherein the process is performed at a temperature from about 100 degrees Celsius to about 200 degrees Celsius.

Aspect A34. The process of any one of aspects A19-A33, wherein the process is performed in the absence of a solvent.

Aspect A35. A compound comprising one or more optionally substituted 1-carboxamide imidazole moieties formed by the process of any one of aspects A19-A34.

### Additional Alternative Aspects

Aspect B1. A process for depolymerizing a polyester comprising treating the polyester with an optionally substituted imidazole to form one or more products comprising one or more optionally substituted 1-acyl imidazole moieties.

Aspect B2. The process of aspect B1, wherein the polyester was formed by condensation of a dicarboxylic acid and a polyol.

Aspect B3. The process of aspect B1 or B2, wherein the polyester is selected from poly(ethylene terephthalate) (PET), poly(trimethylene terephthalate) (PTT), poly(butylene terephthalate) (PBT), poly(ethylene isophthalate), poly(octamethylene terephthalate), poly(decamethylene terephthalate), poly(pentamethylene isophthalate), poly(butylene isophthalate), poly(hexamethylene isophthalate), poly(hexamethylene adipate), poly(pentamethylene adipate), poly(pentamethylene sebacate), poly(hexamethylene sebacate), poly(1,4-cyclohexylene terephthalate), poly(1,4-cyclohexylene sebacate), poly(ethylene terephthalate-co-sebacate), and poly(ethylene-co-tetramethylene terephthalate).

Aspect B4. The process of any one of aspects B1-B3, wherein the one or more products comprise one or more 1-acyl imidazole derivatives of terephthalic acid, isophthalic acid, 2,6-naphthalene dicarboxylic acid, 4,4′-diphenylether dicarboxylic acid, hexahydrophthalic acid, 2,7-naphthalenedicarboxylic acid, phthalic acid, 4,4′-methylenebis(benzoic acid), oxalic acid, malonic acid, succinic acid, methyl succinic acid, glutaric acid, adipic acid, 3-methyladipic acid, pimelic acid, suberic acid, azelaic acid, sebacic acid, 1,11-undecanedicarboxylic acid, 1,10-decanedicarboxylic acid, hexadecanedioic acid, docosanedioic acid, tetracosanedioic acid, 1,4-cyclohexanedicarboxylic acid, 1,3-cyclohexanedicarboxylic acid, 1,2-cyclohexanediacetic acid, fumaric acid, maleic acid, or combinations thereof.

Aspect B5. The process of any one of aspects B1-B4, wherein the one or more products further comprise one or more polyols.

Aspect B6. The process of aspect B1, wherein the polyester was formed by condensation of a hydroxy-substituted acid or ester or derivative thereof, such as a lactone.

Aspect B7. The process of aspect B1 or B6, wherein the polyester is selected from polyglycolic acid (PGA), polylactic acid (PLA), polycaprolactone (PCL), polyhydroxybutyrate (PHB), or a polyhydroxyalkanoate (PHA).

Aspect B8. The process of any one of aspects B1 or B6-B7, wherein the one or more products comprise one or more 1-acyl imidazole derivatives of glycolic acid, lactic acid, hydroxycaproic acid, hydroxybutyric acid, or a hydroxyalkanoic acid, or combinations thereof.

Aspect B9. The process of aspect B1, wherein the polyester comprises polyethylene terephthalate (PET).

Aspect B10. The process of any one of aspects B1-B9, wherein the optionally substituted imidazole comprises a compound of Formula I

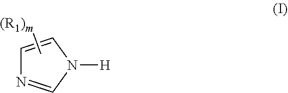

wherein
m is 0, 1, 2, or 3; and
R₁ is independently selected at each occurrence from hydrogen, halo, cyano, azido, nitro, silyl, hydroxy, amino, aldehyde, carboxylic acid, ester, C₁-C₆ alkyl, C₁-C₆ haloalkyl, C₂-C₆ alkenyl, C₂-C₆ alkynyl, C₁-C₆ alkoxy, C₃-C₆ cycloalkyl, 3- to 8-membered monocyclic or bicyclic heterocycle, 6- to 10-membered monocyclic or bicyclic aryl, 5- to 10-membered monocyclic or bicyclic heteroaryl, sulfo-oxo, sulfonyl, sulfone, sulfoxide, sulfonylamino, and thiol.

Aspect B11. The process of any one of aspects B1-B10, wherein the optionally substituted imidazole is selected from imidazole, 2-ethylimidazole, 2,4,5-trimethylimidazole, 2-isopropylimidazole, and 4-methylimidazole.

Aspect B12. The process of any one of aspects B1-B11, wherein the optionally substituted imidazole is present in an amount of about 2 molar equivalents to about 10 molar equivalents relative to the polyester.

Aspect B13. The process of any one of aspects B1-B12, wherein the process is performed in the presence of a base.

Aspect B14. The process of aspect B13, wherein the base comprises a hydroxide or a carbonate.

Aspect B15. The process of aspect B13 or B14, wherein the base comprises sodium hydroxide or potassium hydroxide.

Aspect B16. The process of any one of aspects B13-B15, wherein the base is present in an amount of about 0.05 molar equivalents to about 0.6 molar equivalents relative to the polyester.

Aspect B17. The process of any one of aspects B1-B16, wherein the process is performed at a temperature from about 100 degrees Celsius to about 200 degrees Celsius.

Aspect B18. The process of any one of aspects B1-B17, wherein the process is performed in the absence of a solvent.

Aspect B19. A compound comprising one or more optionally substituted 1-acyl imidazole moieties formed by the process of any one of aspects B1-B18.

Aspect B20. A process for depolymerizing a polyurethane comprising treating the polyurethane with an optionally substituted imidazole to form one or more products having one or more optionally substituted 1-carboxamide imidazole moieties.

Aspect B21. The process of aspect B20, wherein the polyurethane was formed by condensation of one or more diisocyanates, one or more polyols, optionally one or more chain extenders, and optionally one or more crosslinking agents.

Aspect B22. The process of aspect B21, wherein the one or more diisocyanates are selected from an aliphatic diisocyanate, an aromatic diisocyanate, or combinations thereof.

Aspect B23. The process of aspect B21, wherein the one or more diisocyanates are selected from 4,4′-dicyclohexylmethane diisocyanate, isophorone diisocyanate, 4,4′-diphenylmethane diisocyanate, 2,4-toluene diisocyanate, 2,6-toluene diisocyanate, hexamethylene diisocyanate, or combinations thereof.

Aspect B24. The process of aspect B21, wherein the one or more polyols are selected from a polyether polyol, a polycarbonate polyol, a polyester polyol, a silicone polyol, or combinations thereof.

Aspect B25. The process of aspect B21, wherein the one or more polyols are selected from polyethylene glycol, polypropylene glycol, polytetramethyleneglycol, poly(1,6-hexamethylene carbonate)diol, poly(decamethylene carbonate)diol, oligocarbonate diol, poly(hexamethylene-pentamethylene carbonate)diol, polylactide, polycaprolactone, polyethylene glycol adipate, polydimethylsiloxane, polyaryl siloxane, and polyalkyl siloxane.

Aspect B26. The process of any one of aspects B20-B25, wherein one or more products comprise one or more 1-carboxamide imidazole derivatives of 4,4′-dicyclohexylmethane diisocyanate, isophorone diisocyanate, 1,4-cyclohexylmethane diisocyanate, 4,4′-diphenylmethane diisocyanate, 2,4-toluene diisocyanate, 2,6-toluene diisocyanate, hexamethylene diisocyanate, or combinations thereof.

Aspect B27. The process of any one of aspects B20 to B26, wherein the one or more products further comprises one or more polyols.

Aspect B28. The process of any one of aspects B20-B27, wherein the optionally substituted imidazole comprises a compound of Formula I

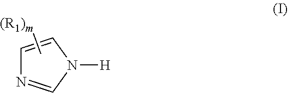

wherein
m is 0, 1, 2, or 3; and
R₁ is independently selected at each occurrence from hydrogen, halo, cyano, azido, nitro, silyl, hydroxy, amino, aldehyde, carboxylic acid, ester, C₁-C₆ alkyl, C₁-C₆ haloalkyl, C₂-C₆ alkenyl, C₂-C₆ alkynyl, C₁-C₆ alkoxy, C₃-C₆ cycloalkyl, 3- to 8-membered monocyclic or bicyclic heterocycle, 6- to 10-membered monocyclic or bicyclic aryl, 5- to 10-membered monocyclic or bicyclic heteroaryl, sulfo-oxo, sulfonyl, sulfone, sulfoxide, sulfonylamino, and thiol.

Aspect B29. The process of any one of aspects B20-B28, wherein the optionally substituted imidazole is selected from imidazole, 2-ethylimidazole, 2,4,5-trimethylimidazole, 2-isopropylimidazole, and 4-methylimidazole.

Aspect B30. The process of any one of aspects B20-B29, wherein the optionally substituted imidazole is present in an amount of about 2 molar equivalents to about 10 molar equivalents relative to the polyurethane.

Aspect B31. The process of any one of aspects B20-B30, wherein the process is performed in the presence of a base.

Aspect B32. The process of any one of aspect B31, wherein the base comprises a hydroxide or a carbonate.

Aspect B33. The process of aspect B31 or B32, wherein the base comprises sodium hydroxide or potassium hydroxide.

Aspect B34. The process of any one of aspects B20-B33, wherein the base is present in an amount of about 0.05 molar equivalents to about 0.6 molar equivalents relative to the polyurethane.

Aspect B35. The process of any one of aspects B20-B34, wherein the process is performed at a temperature from about 100 degrees Celsius to about 200 degrees Celsius.

Aspect B36. The process of any one of aspects B20-35, wherein the process is performed in the absence of a solvent.

Aspect B37. A compound comprising one or more optionally substituted 1-carboxamide imidazole moieties formed by the process of any one of aspects B20-B36.

A number of aspects of the disclosure have been described. Nevertheless, it will be understood that various modifications may be made without departing from the spirit and scope of the invention. Accordingly, other aspects are within the scope of the following claims.

By way of non-limiting illustration, examples of certain aspects of the present disclosure are given below.

### EXAMPLES

The following examples are set forth below to illustrate the compounds, compositions, and methods claimed herein, along with associated methods and results according to the disclosed subject matter. These examples are not intended to be inclusive of all aspects of the subject matter disclosed herein, but rather to illustrate representative methods and results. These examples are not intended to exclude equivalents and variations of the present disclosure, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1. Imidazolysis of PET and PU

This example details a method for chemically upcycling poly(ethylene terephthalate) (PET) and poly(urethane) (PU) materials. An efficient synthetic approach was developed to selectively cleave PET and PU polymer chains at the —O— linkage of the ester or urethane linkage via imidazolysis, a form of aminolysis using the N-based heterocycle, imidazole. Imidazole (8 eq.) serves as a reactant and solvent, and when mixed with PU tubing or PET bottle waste plastics (1 eq.) and catalytic base (i.e. 0.5 eq. NaOH) in a round-bottom heavy walled pressure vessel and then heated to 140-170° C. while stirring. This reaction cleaves the polymer into its constituent building blocks, including carboxamide or carbonyl bis(imidazoles) (i.e. 1,4-phenylenebis((1H-imidazol-1-yl)methanone) or N,N′-(methylenebis(4,1-phenylene))bis(1H-imidazole-1-carboxamide)), glycol byproducts, and residual imidazole. At elevated temperature, the imidazole melts and allows for stirring; however, the high temperature of 140° C. for the PU reaction and 170° C. for the PET reaction is required to fully dissolve the raw polymers. Following the reaction even at high temperatures (before cooling), the product has precipitated as a yellow-orange solution and tan solid, respectively.

The PU+Imidazole reaction was precipitated in DI H₂O, followed by vacuum filtration and washing with cold acetone to yield the product N,N′-(methylenebis(4,1-phenylene))bis(1H-imidazole-1-carboxamide) as a tan solid powder. The PET+Imidazole reaction should not be precipitated in DI H₂O, as the carbonyl-imidazole compound and all reactants solubilize when contacted with H₂O. Instead, this product was filtered, and then stirred in Et₂O for 24 h before isolation via vacuum filtration to yield the product 1,4-phenylenebis((1H-imidazol-1-yl)methanone as an off-white solid powder. These reactions are scalable, and have been successful when run with a 5-10 g basis of the PET or PU.

The glycol products may be recoverable. While other bases were tried (i.e. K₂CO₃), NaOH was the most efficient, yielding the bis(imidazole) products in yields >70%. The bis(imidazole) products have the potential to be repolymerized (for example, via the Menshutkin reaction or substitution reactions with a difunctional species) or participate in a variety of chemistries following this depolymerization into discrete, functional molecules.

### Example 2. Depolymerization of Polyurethane

### Depolymerization Stages

1. Grind the commercial grade polyurethane into finer particles for more efficient and homogeneous degradation reaction
2. React polyurethane with imidazole-derivatives in a microwave-assisted synthesis reactor
3. The solid product obtained is extracted with an effective solvent (e.g., hexane).
4. Evaporate and recycle the solvent
5. Collect the remaining polyol products
6. Purify the polyols

### Characterization

### FT-IR of Polyurethane Reaction Products

The FT-IR spectra support the existence of polyols as we get the hydroxyl OH⁻ group at the expected region for the PU reacting with imidazole and 2-methyl-imidazole. Additionally, the urethane (C═O) at 1700 cm⁻¹ and amide linkage (NH) at 3245 cm⁻¹ in the original PU is not visible in the expected polyols from the reaction of PU with imidazole and 2-methylimidazoles.

### NMR of Polyurethane Reaction Products

Protons on the carbon adjacent to the aliphatic alcohol appear in the region of 3.4-5.00 ppm. We have observed a peak at this position for the reaction of PU with imidazole and 2-methyl-imidazole.

### Example 3. Depolymerizing PET Via Imidazolysis for Obtaining a Diverse Array of Intermediates from Plastic Waste

Polyethylene terephthalate (PET) is a ubiquitous commodity plastic used in applications including textiles, food packaging, drink bottles, and thermoplastic resins. Like other synthetic polymers, the massive accumulation of PET on Earth's surface has presented formidable environmental challenges. As a polyester, PET is susceptible to chain cleavage (i.e., depolymerization) via various “chemolysis” methods. In this Example, we introduce an approach to PET cleavage by imidazole (and related compounds): “imidazolysis”. Reacting PET with excess imidazole yields 1,1′-terephthaloylbisimidazole (TBI) which can be further transformed into an array of small products such as amides, benzimidazoles, and esters, or potentially used as monomers for polymers. The TBI molecules obtained via imidazolysis are versatile intermediates (owing to their activated carbonyl groups) which can be stored and subsequently converted to specific final products later. This means that the target product(s) do not have to be predetermined when the depolymerization reaction is carried out, and this methodology could provide flexibility to meet demands for various chemical products based on the terephthalic acid (or p-xylene) motif. Imidazolysis may also be of broad utility in depolymerizing other polyesters as well as polyurethanes (PU).

### Introduction

The practice of recycling synthetic plastics is perceived to be a socially responsible task in modern times as it plays a crucial role in collecting useful materials, combatting environmental pollution, and conserving petroleum reserves. However, the majority of plastics are still either landfilled or incinerated.

The commercial and household consumption of poly(ethylene terephthalate) (PET) has experienced significant growth in recent decades, particularly from the production of bottles and containers for food, drinks, and personal care products, as well as for textile fibers, and other consumer goods.¹ The widespread adoption of PET can be attributed to its exceptional durability, gas barrier properties, cost-effectiveness, minimal need for processing additives, etc.² PET has an annual global production of ˜70 Mton, with 71% of this production dedicated to plastic bottle manufacturing.³⁻⁵ While there is a well-established collection and sorting infrastructure for handling post-consumer PET,⁶ the overall recycling rate for PET waste remains relatively low, averaging ˜29% in the United States.⁷

The simplest approach to recycling plastic is through thermomechanical processing; however, this is better considered to be “downcycling” as it detrimentally affects polymer properties, yielding materials with fewer uses and less value than “virgin” plastics.⁸ Because of these challenges, chemical recycling of plastics (i.e., depolymerization) has gained significant interest since it targets reducing waste while still taking advantage of the chemistry of plastics.⁹ Notably, PET is the only condensation polymer among the plastics stamped with resin identification codes (RIC) 1-6. PET, and more generally, the polyalkylene terephthalate (PAT) family are polyesters that are susceptible to attack/degradation via “chemolysis” with various reagents.

Thus far, significant advancements have been made in the depolymerization and partial chemical modification of PET through chemolysis methods, including hydrolysis (water),^{10, 11} alcoholysis (alcohols),^{12, 13} glycolysis (ethylene glycol),^{9, 14, 15} aminolysis (amines),^{5, 8, 16} and ammonolysis (ammonia).^{9, 17} Each technique involves chemically cleaving an ester bond with the corresponding nucleophile, leading to the formation of C—O or C—N bonds.

Some chemolysis approaches to PET are targeted at recovering essential building blocks for the synthesis of fresh PET such as dimethyl terephthalate (DMT). DMT is obtained through alcoholysis with MeOH. Recovery of terephthalic acid (TA) can be achieved via hydrolysis.^{10, 13} Furthermore, the glycolysis of PET to bis-2-hydroxyethyl terephthalate (BHET) has already been successfully implemented on an industrial scale.^{18, 19} In recent years, aminolysis has emerged as a rapid and economical method for PET depolymerization, owing to the more reactive nature of amines, as the conversion of esters to amides can occur even at ambient temperature. For example, the quantitative depolymerization of PET by primary (1°) amines or amino alcohols such as monoethanolamine (MEA) can be carried out at ambient temperature in the absence of a cosolvent using sufficient molar excess (e.g., 20 eq.) of the amine yielding terephthalamide diols. Low to moderate molecular weight poly(ester-amide)s have been successfully synthesized from these terephthalamide diols at lab-scale quantities (FIG. 5A), and they have utility as biodegradable polymers in tissue engineering.^{8, 16, 20, 21} However, broader applications for small terephthalamide molecules (e.g., N,N′-dibutylterephthalamide) have not yet emerged.

The aminolysis of PET using 1-(3-aminopropyl)imidazole (API) has been previously reported, yielding a bis-imidazole terephthalamide (FIG. 5B), which we had previously synthesized using API and terephthaloyl chloride in acetonitrile (CH₃CN) with potassium carbonate (K₂CO₃) as a base.²² This product was used to synthesize imidazolium polyamide-ionenes, which have intrinsic and rapid self-healing properties and are highly amenable to 3D printing.^{22, 23}

It is worth noting that the depolymerization of PET by API is still a classical aminolysis of an ester; the imidazole moiety did not participate in the reaction. However, that work has led us to consider whether imidazole itself might be able to depolymerize PET via “imidazolysis”. Imidazole is conventionally represented as having both secondary (2°) and tertiary (3°) amines within the 5-membered heterocycle, although the true structure is a tautomer.^{24, 25} As demonstrated herein, imidazole and its analogs such as 2-methylimidazole, 4-methylimidazole, etc., can cleave esters forming 1,1′-terephthalolylbisimidazoles (TBIs, FIG. 5C) while 1-methylimidazole (N-methylimidazole) would not.

Imidazole and its derivatives find use in both natural and synthetic organic chemistry. Imidazole moieties are found in pharmaceuticals (e.g., miconazole, clotrimazole), fungicides (e.g., prochloraz) and biologically significant compounds (e.g., histidine, an essential amino acid).^{26, 27} Imidazole is also a building block for N-alkylimidazoles,²⁸ imidazolium ionic liquids (ILs), ionic polymers. The versatility of imidazoles for organic synthesis arises from the ability to create heterocycles with many different substituents on the N and/or C atoms within the 5-membered ring.^{25, 29-31} A recent work has demonstrated the role of imidazoles as a catalyst in obtaining a reaction intermediate which enhances the selective depolymerization of bisphenol A-based polycarbonate (BPA-PC) into valuable cyclic complex carbonates and BPA under mild condition.³²

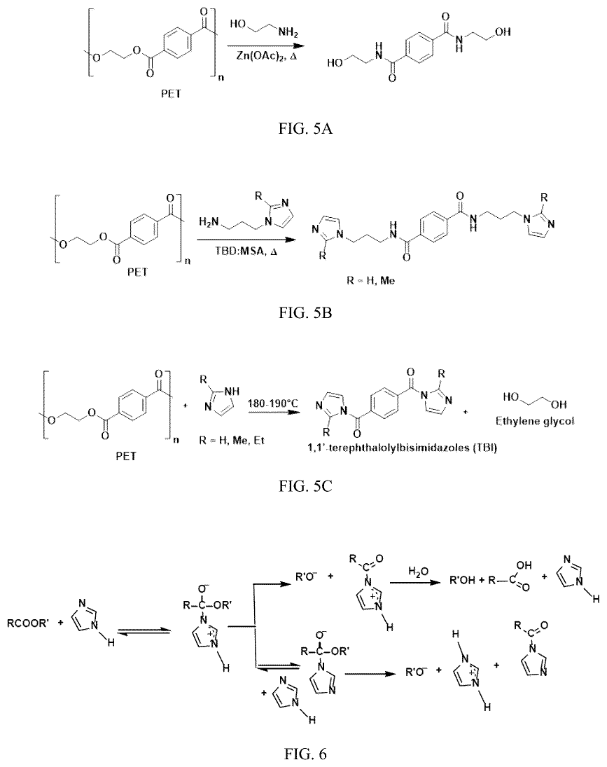

In this example, we describe the utility of imidazole compounds in depolymerizing PET and the subsequent reactions of those products to form small molecules. During the preparation of this example, we searched SciFinderⁿ for the term “imidazolysis,” which yielded a total of 9 results, with publication dates ranging from 1959 to 2003. In 1952, Boyer reported on the acetylation of imidazole via acid (H⁺) catalyzed reaction with isopropenyl acetate but did not use the term “imidazolysis”. Scheme 1a illustrates how the final product was balanced by the enol to keto tautomerization from reactant to acetone, except for another product, “1-acetylimidazole” in this reaction.³³ In 1998, Schmeer investigated the imidazolysis of esters in the presence of different solvents (water, acetonitrile, 1,4-dioxane, and propylene carbonate, etc.); where the formation of tetrahedral zwitterionic as an intermediate which later dissociates into two different routes (FIG. 6).³⁴ Later, in 1977, Rivetti and Tonellato further investigated the imidazolysis of esters in benzene. They used an equimolar solution of p-nitrophenyl propionate (PNPP) and imidazole, which resulted in a reversible reaction at room temperature (Scheme 1b).³⁵ However, we did not find imidazolysis of small diesters in the existing literature.

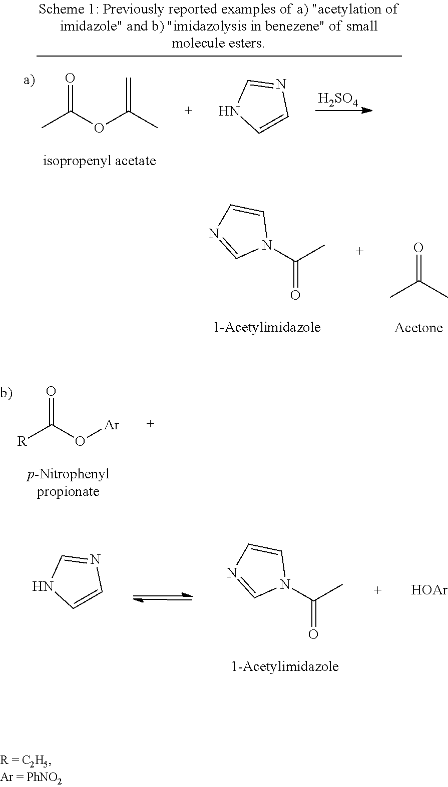

Herein, we report the quantitative depolymerization of PET (or any polyester) via imidazolysis. In this example, imidazolysis was performed without a cosolvent, as the melting points of the imidazole compounds are sufficiently low to serve as both reactant and solvent. The obtained symmetric TBI products are connected to the broader family of N-acyl imidazoles (Scheme 2), which are well-known exclusive electrophiles that exhibit tunable reactivity and chemical selectivity towards forming esters and amides favoring nucleophilic substitution reactions.^{36, 37}

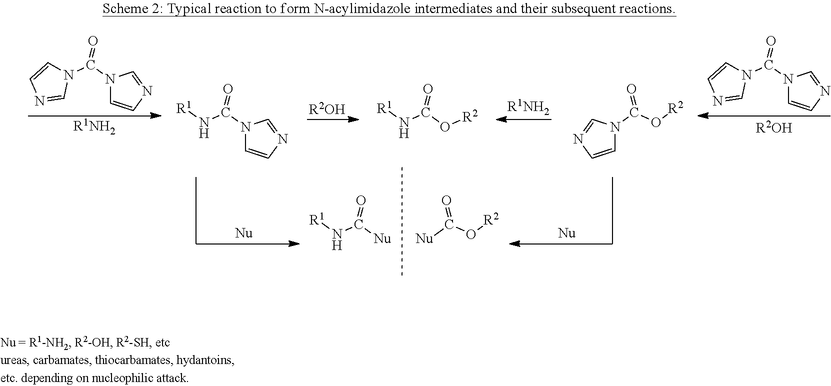

N-acylimidazoles are generally produced from N,N-carbonyldiimidazole (CDI), which itself is typically obtained via the reaction of excess imidazole with phosgene (COCl₂). CDI is commonly used for coupling amino acids for peptide synthesis but has also been demonstrated to be useful a functionality within polymer science for synthesizing urethane and urea-containing monomers, as explained in Scheme 2.^{38, 39}

Staab has already reviewed the importance of azolides (N-heterocyclic amides, e.g., CDI, N-acylimidazoles) of carbonic acid derivatives in transacylation reactions, where the high degree of reactivity of azolides results from the quasi-aromatic nature of azoles.⁴⁰ Following these, diimidazolides or TBIs can also show equivalent performance. Therefore, the molecules obtained via imidazolysis of PET (and likely other polyesters) are sufficiently reactive to provide a versatile platform to generate amides and esters based on the choice of amines and alcohols, respectively (Scheme 3). The TBIs derived from waste PET are functionally equivalent to terephthaloyl chloride or terephthalaldehyde. Thus they can be used to obtain small molecules like bisbenzimidazoles, diamides, diesters, and imidazolines/oxazolines,^{22, 31} and potentially polymers. These findings underscore the ability of “imidazolysis” as a means of developing intermediates with enhanced properties and promote a circular economy for plastic materials.

### Experimental Details

### Materials

Imidazole (99%) and o-Phenylenediamine (o-PDA, 99%) were purchased from Beantown Chemical (Hudson, NH, USA). Polyphosphoric acid (PPA, >85%), Phosphoric acid (H₃PO₄, 99%), and 2-Ethylimidazole (99%) were purchased from Thermo Scientific (Waltham, MA, USA). 2-Ethyl-4-Methylimidazole (96.82%) was purchased from Chem-Impex Int'l Inc. (Wood Dale, IL, USA). 2-Isopropylimidazole (98%) and 4-Methylimidazole (>98%) was purchased from Tokyo Chemical Industry Co., Ltd (Portland, OR, USA). 2-Phenylimidazole (>98%), n-Butylamine (99%), and 2-Methylimidazole (97%) were purchased from Alfa Aesar (Ward Hill, MA, USA). All these chemicals were used as received except o-PDA, which was recrystallized prior to use. Acetonitrile (ACN), Tetrahydrofuran (THF), and Acetone were used as solvents and purchased from VWR. 2,4,5-Trimethylimidazole was previously synthesized in our laboratory according to a published method.⁴¹

### Experimental Procedure

Post-consumer PET bottles were collected and thoroughly washed with soapy water, followed by thorough rinsing with deionized water. Acetone was then used to remove any remaining surface contaminants. PET bottles were manually cut into 5-6 mm flakes and dried in an oven at 70° C. overnight. The depolymerization reactions were initially performed at small scales (e.g., 2 g PET (10.4 mmol), 5.6 g imidazole (83.2 mmol)) in a microwave synthesis reactor (MCR). A predetermined weight of PET (pellets/flakes) and 8 eq. of imidazole (per PET repeat unit) were thoroughly homogenized together via grinding (IKA Multidrive Control) before loading the mixture into a 30 mL microwave reaction vial. This co-grinding of PET+imidazole was found to yield much more consistent results as the PET and imidazole were already in intimate contact prior to heating. A Monowave 400 microwave reactor (Anton Paar GmbH, 1600 VA, 850 W maximum magnetron power output) was used with temperature, time, and stirring speed control. PET depolymerization with selected imidazole derivatives (e.g., imidazole, 2-methylimidazole) was also successfully scaled up (e.g., 35 g PET with 110 g imidazole) in a 250 mL heavy-walled round-bottom pressure vessel (Ace Glass).

The disappearance of the PET particles was visually monitored, and heating was continued until the mixture became homogenous. After this, the reaction mixture was allowed to cool to room temperature overnight. ACN was added to the crude mixture, allowing for the removal of any possible remaining PET oligomers, excess imidazole, and the produced ethylene glycol (EG). The TBI products were insoluble in ACN, and this allowed for them to be collected via vacuum filtration, where they were washed with THF. The TBI products were off-white or greyish solid powders for compounds 1b to 4b and yellowish to dark brown solid powders for compounds 5b to 8b. These products were dried under vacuum at 75° C. overnight.

### Computational Methods

Density functional theory (DFT) calculations were used to analyze the electrostatic potential (ESP) features of the imidazole molecules in the gas phase; explicitly, we evaluated PET-Imidazole (SM1), PET-2-Methyl-imidazole (SM2), PET-3-Methyl-imidazole (SM3), and (1H-imidazol-1-yl)(phenyl)methanone (SM4). Initially, 40 different conformers were generated using the generic algorithm of OpenBabel,⁴² followed by geometric optimization using the B3LYP functional^{43, 44} and a 6-31(d) basis set.^{45, 46} Each of the lowest energy conformers was selected for further electrostatic surface potential (ESP) and Conceptual Density Functional Theory (CDFT) analyses.⁴⁷⁻⁵⁰ To do this, a single point energy calculation of the chosen conformer was conducted using a 6-31++(d,p) basis set,⁵¹ along with DFT-D3 dispersion corrections.⁵² The Multiwfn package⁵³ was used to calculate the ESP and general interaction properties functions (GIPFs).⁵⁴⁻⁵⁶ The GIPFs were determined utilizing van der Waals surfaces and an electron density isosurface of 0.001 e/Bohr³.⁵⁵ Based on this approach, we obtained the molecular volume (Vₘ), surface area (SA), minimum ESP value (Vₘᵢₙ), maximum ESP value (Vₘₐₓ), average ESP value (V), and molecular polar index (MPI) of each molecule.

To further analyze the chemical stability of the evaluated molecules, CDFT properties such as the vertical ionization potential (VIP), vertical electron affinity (VEA), Mulliken electronegativity (χ), chemical potential (μ), hardness (η), softness (η⁻¹), and Parr's electrophilicity index (ω) were calculated using the wave function of N, N−1, and N+1 electron states, in which N is the number of electrons corresponding to the neutral molecule. In our approach, CDFT properties were also calculated using the Multiwfn package, and the single-point energy calculations were performed using the 6-31++(d,p) basis set and DFT-D3 dispersion corrections. Finally, we inferred the relative N—C bond strength by carrying out incremental scans corresponding to different fixed bond lengths, in which the energy of each conformer is plotted as a function of the distance between the C and N bonded sites, carried out using the same level of theory described above with an increment spacing of 0.1 Å. All DFT calculations were performed using Gaussian 09.⁵⁷

### Results and Discussion

### Depolymerization of PET Via Imidazolysis

Imidazole itself, along with alkyl or aryl-substituents at the 2, 4, and/or 5 positions, are comparable to heterocyclic 20 amines, exhibiting moderate basicity (pKₐ=7 to 8.92) (Table 2) and are able to undergo nucleophilic N-substitution reactions with esters, as depicted in Scheme 3 and FIG. 5C. This study screened the depolymerization of PET via imidazolysis using eight different imidazoles shown in Table 1 and their comparative reactivity toward forming the corresponding symmetric TBI compounds.

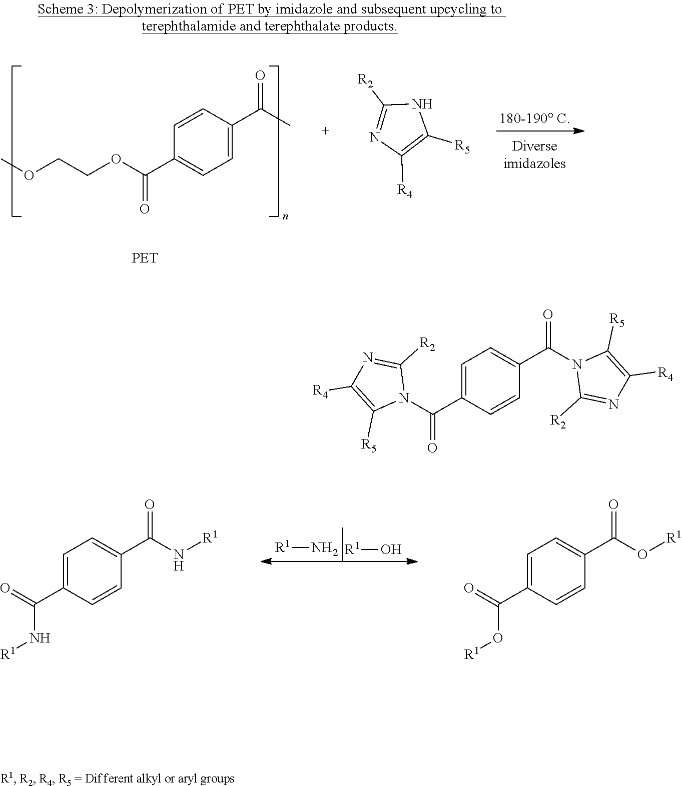

TABLE 1
Imidazole reagents used, and corresponding products obtained
via imidazolysis of PET.

| Reagent | Product |
|---|---|
| 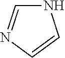 | 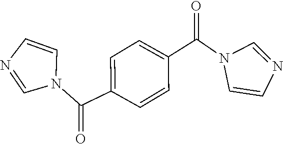 [SMILES: O=C(c1ccc(C(=O)n2ccnc2)cc1)n1ccnc1] |
| Imidazole | 1b |
| 1a | |
| 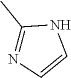 | 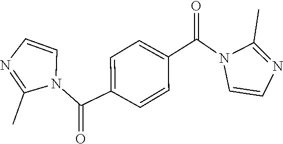 [SMILES: Cc1nccn1C(=O)c1ccc(C(=O)n2ccnc2C)cc1] |
| 2-Methyl | 2b |
| imidazole | |
| 2a | |
| 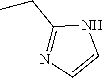 | 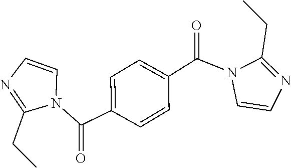 [SMILES: CCc1nccn1C(=O)c1ccc(C(=O)n2ccnc2CC)cc1] |
| 2-Ethyl | 3b |
| imidazole | |
| 3a | |
| 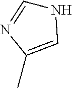 | 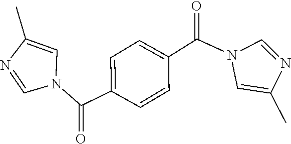 [SMILES: Cc1cn(C(=O)c2ccc(C(=O)n3cnc(C)c3)cc2)cn1] |
| 4-Methyl | 4b |
| imidazole | |
| 4a | |
| 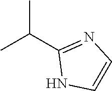 | 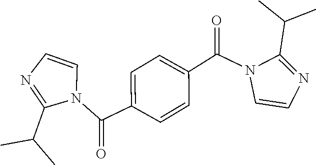 [SMILES: CC(C)c1nccn1C(=O)c1ccc(C(=O)n2ccnc2C(C)C)cc1] |
| 2-Isopropyl | 5b |
| imidazole | |
| 5a | |
| 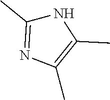 | 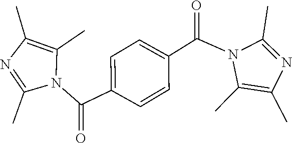 [SMILES: Cc1nc(C)n(C(=O)c2ccc(C(=O)n3c(C)nc(C)c3C)cc2)c1C] |
| 2,4,5- | 6b |
| Trimethyl | |
| imidazole | |
| 6a | |
| 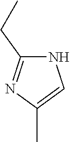 | 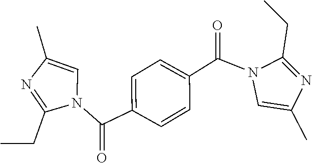 [SMILES: CCc1nc(C)cn1C(=O)c1ccc(C(=O)n2cc(C)nc2CC)cc1] |
| 2-Ethyl-4- | 7b |
| methyl | |
| imidazole | |
| 7a | |
| 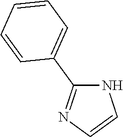 [SMILES: c1ccc(-c2ncc[nH]2)cc1] | 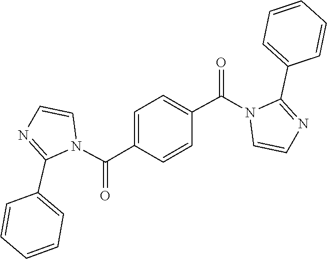 [SMILES: O=C(c1ccc(C(=O)n2ccnc2-c2ccccc2)cc1)n1ccnc1-c1ccccc1] |
| 2-Phenyl | 8b |
| imidazole | |
| 8a | |

Imidazole, possessing moderate basicity (pKₐ˜7)⁵⁸, was first monitored in reaction with PET at 180′° C. Over a duration of 80 min, all of the PET particles completely disappeared, with a recovered yield of 61% of 1,1′-(1,4-Phenylene)bis[1-(1H-imidazol-1-yl)methanone] (“TBI”, 1b). The product was verified by ¹H NMR and ¹³C NMR spectra.

Given the success with imidazole, reactions with imidazole derivatives bearing a single alkyl substituent were then considered (2a-5a). The presence of electron-donating (e.g., -Me, -Et) groups on the imidazole ring resulted in faster reactions. It has been established in prior findings that the basicity of amines is highly correlated with their reactivity in amidation reactions with esters.¹⁶ As such, we obtained a more substantial yield (95%) of product 2b, which suggests 2-methylimidazole is a more effective imidazolysis reagent for PET depolymerization. This correlates with the larger pKₐ of 2-methylimidazole (pKₐ=8.1). However, it is worth noting that the melting point of 2-methylimidazole (Tₘ=142° C.) is significantly higher than that of imidazole (Tₘ=86° C.) such that the reaction was run at a slightly increased temperature (190° C.) primarily to reduce viscosity. Reactions of PET with 3a and 4a were also successful in obtaining TBIs with acceptable yields (see Table 2). The imidazolysis of PET with 2-isopropylimidazole (5a) was also attempted but was found to have a lower yield despite the stronger basicity of 5a than imidazole. One possible reason could be the steric hindrance caused by the isopropyl group at the same position, affecting the progress of the reaction even at 190° C. These characteristics are also reflected in the solubility of product 5b with common organic solvents (Table 3).

TABLE 2
Parameters for imidazolysis reactions of PET and yield of recovered product.

| | | | | | Depolymerization | | | |
|---|---|---|---|---|---|---|---|---|
| | | | | | (MCR data) | | | |
| | | Tₘ | | | Temp | Time | Yield^{c} | Yield^{d} |
| Entry^{a} | Imidazole | (° C.)^{b} | pKₐ⁵⁸ | Product | (° C.) | (min) | (%) | (%) |
| 1 | Imidazole (1a) | 85-92 | 7 | 1b | 180 | 80 | 61 | 82 |
| 2 | 2-Methyl-imidazole | 142-144 | 7.85-8.10 | 2b | 190 | 60 | 70 | 95 |
| | (2a) | | | | | | | |
| 3 | 2-Ethyl-imidazole | 78-81 | 7.99-8 | 3b | 180 | 80 | 66 | 71 |
| | (3a) | | | | | | | |
| 4 | 4-Methyl-imidazole | 44-47 | 7.69-7.80 | 4b | 180 | 90 | 39 | 65 |
| | (4a) | | | | | | | |
| 5 | 2-Isopropyl-imidazole | 129-131 | 7.97 | 5b | 190 | 100 | 38 | — |
| | (5a) | | | | | | | |
| 6 | 2,4,5- | 65^{d} | 8.92⁵⁹ | 6b | 60 | 60 | 72 | — |
| | Trimethylimidazole | | | | | | | |
| | (6a) | | | | | | | |
| 7 | 2-Ethyl 4-methyl | 47-54 | 8.68 | 7b | 190 | 135 | 34 | 60 |
| | imidazole (7a) | | | | | | | |
| 8 | 2-Phenyl-imidazole | 142-148 | 6.44⁶⁰ | 8b | 180 | 120 | 32 | 59 |
| | (8a) | | | | | | | |

^{b}Melting point according to consumer product,
^{c} = yield obtained by pressure vessel reaction,
^{d} = determined in the lab
^{a}2 g PET and 8 eq. (in mole) of imidazole were used for microwave reaction. 10 g PET with 8 eq. of imidazole compounds was used for upscaling in a pressure vessel reaction (For reagent 1a, PET was taken as 35 g).

Considering that the basicity of imidazole can be influenced/enhanced by the position and size of alkyl groups on the C(2), C(4), and/or C(5) positions of the imidazole ring structure, di- and tri-alkyl-substituted reagents were also tested (6a, 7a).⁵⁸ With these compounds, some variation in reaction parameters was required to achieve depolymerization, although each has a comparable basicity (Table 2). Product 6b was obtained at 180° C. with the shortest reaction time (30 min) and a good yield (72%), which can be attributed to the high nucleophilicity of 2,4,5-trimethylimidazole (6a) (pKₐ=8.92). It is worth noting that an exception was observed for 2-ethyl-4-methylimidazole (7a), as the imidazolysis of PET proceeded slowly even at 190° C., taking almost 2.5 h to complete in the microwave reactor. Similar to 5a, it is hypothesized that the steric bulk of 7a could lead to poor interaction with PET flakes, making it an ineffective reagent for depolymerization, and one additional cause might be its lower basicity (pKₐ=8.68) compared to 6a. The presence of all these monomers had been confirmed by ¹H and ¹³C NMR.

Finally, 2-phenylimidazole (8a) was considered for the imidazolysis of PET. This compound contains a pendant benzene ring, which has the effect of making it the weakest base (pKₐ=6.44) among all of the imidazoles considered. However, it was observed that 8a was able to fully depolymerize PET, although it required 120 min. The bulky phenyl group and a high melting point of 8a might be responsible for the slow reaction. It is worth mentioning that product 8b was moderately soluble in typical aromatic organic solvents (e.g., toluene, xylene), distinguishing it from other TBIs (Table 3).

The TBI produced (1b-4b) in these reactions exhibits specific solubility (1 mg TBI per mL of solvent) in common alcohols (e.g., MeOH, EtOH) and water, and these solvents do not react with TBI molecules at room temperature. The solubility of all the TBI products obtained is provided in Table 3.

TABLE 3
Solubility of TBIs in common solvents

| Imidazole | Solvent | | | | | |
|---|---|---|---|---|---|---|
| Compounds | DI Water | Ethanol | DCM | Toluene | Xylene | DMSO |
| 1b | s | s | i | i | i | s |
| 2b | s | s | i | i | i | s |
| 3b | s | s | i | i | i | s |
| 4b | s | s | i | i | i | s |
| 5b | i | i | i | i | i | ps |
| 6b | s | s | i | i | i | s |
| 7b | s | ps | i | i | i | s |
| 8b | i | i | ps | ps | ps | ps |

s = soluble;
i = insoluble;
ps = partially soluble

Overall, these studies show that PET can indeed be depolymerized directly in the melt phase via imidazolysis with different imidazoles, yielding TBI products. The use of a MCR was found to be particularly useful for rapidly screening reagents and reaction conditions before scale-up in sealed pressure vessels. In the MCR, the complete disappearance of PET required between 1.0-2.5 h but resulted in a lower yield. This phenomenon aligns with previous studies where fast heating rates in MCR led to incomplete conversion of some starting materials.⁶¹ In contrast, the traditional reaction with larger PET flakes takes a comparatively longer time but results in a higher yield. Although, the steric bulk on some alkyl-substituted imidazoles can require additional time.

A recent study has shown successful depolymerization of PET and other polyesters with morpholine in the presence of a titanium-based catalyst. The morpholine-amide product was then transformed into valuable small molecules via hydrolysis, Grignard reaction, etc. However, even in the presence of a catalyst, the process required 2 d for complete conversion (>99%).⁶² This example reveals that imidazolysis by conventional heating could make an 82% yield of 1b within a 24 h reaction time without a catalyst. Here, we focused on determining the efficacy of imidazole compounds for the degradation of PET. Future work will consider the possible synergistic effects of catalysts such as TBD:MSA for imidazolysis, which will likely require fewer equivalents of the imidazole compounds and may improve the yields for the bulkier imidazoles.^{8, 22} Hydrolyzing waste PET to terephthalic acid (TA) is a valuable practice in promoting plastic circularity and avoiding the reliance on virgin resources. This work successfully converted back TBI products (1b and 2b) into TA via acid-catalyzed hydrolysis using HCl/H₂SO₄ as catalysts in mild conditions.

### Fourier-Transform Infrared Spectroscopy (FTIR) Analysis

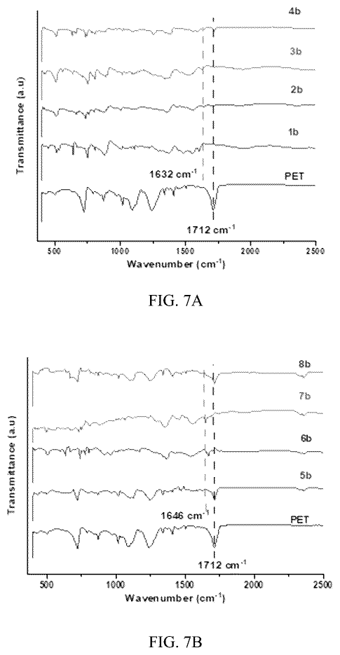

Fourier transfer infrared (FTIR) spectra were obtained with a Thermo Fisher Scientific ATR-FTIR with a resolution of 4 cm⁻¹ and 128 scans within a spectral region of 4000-400 cm^{-1 1}. The frequency of the depolymerized compounds displays distinct and consistent vibrational peaks, which are noticeably different than PET itself (FIGS. 7A-7B). Specifically, the intensity of the sharp ester carbonyl (C═O) peak at 1712 cm⁻¹ in PET decreases in all the depolymerized products. Assigning C═O shifting for TBIs is challenging since these compounds cannot form H-bonds with the adjacent amide molecules and are typically found in the 1670 to 1630 cm⁻¹ region.^{63,64}

However, for N-heterocyclic compounds, this absorption band can be shifted at slightly higher frequencies due to the conjugation between the imidazole ring N(1) and the acyl group, compared to C═O groups attached to aliphatic compounds. Consequently, the symmetric stretching of the acyl imidazoles in the degradation products was found in the range of 1712 to 1600 cm⁻¹, indicating substitutions happened during the reaction. Additionally, there is no trace of characteristic NH—N bonding of unsubstituted imidazole in the 2800 to 2600 cm⁻¹ region, confirming that the TBIs have been formed by the disappearance of the N(1)-H proton. Nevertheless, the spectra still exhibit vibrational frequencies in 1660 to 1450 cm⁻¹ bandwidth in the depolymerized compounds, attributable to intramolecular C—N and C—C bonding.^{64, 65}

### Nuclear Magnetic Resonance Spectroscopy (NMR) Analysis

A Bruker Avance instrument (Billerica, MA) with 500 MHz was used to collect ¹H and ¹³C NMR spectra in DMSO-d₆ for all the TBI products. The product of PET imidazolysis with imidazole (1b) and the requisite proton integrations were analyzed. The most deshielded resonance peak around δ 8 ppm is assigned to the benzene ring, verifying identical para-substitution, and the corresponding carbonyl group is observed near δ=170 ppm in ¹³C NMR. Two signals between δ=7-8 ppm are attributed to the symmetrical imidazole C(2), C(4) and C(5)-H atoms.⁶⁶ At the same time, the presence of C(2) proton is absent in other depolymerized products having 2-substituted imidazoles, and subsequent alkyl groups were evident between δ 1 to 3 ppm in ¹H NMR and at δ 10 to 30 ppm in ¹³C NMR, respectively).

### ESP and CDFT Analysis

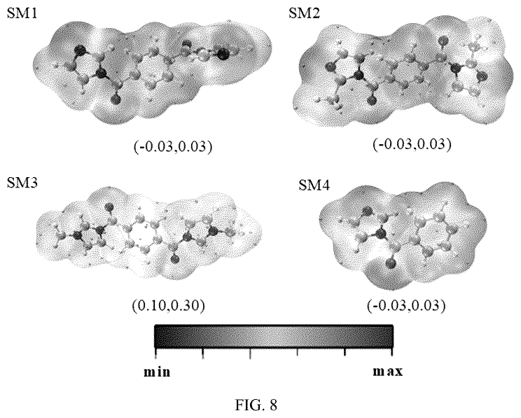

FIG. 8 shows the optimized conformers, and ESP surfaces for the molecules SM1, SM2, SM3, and SM4, in which the red-colored surfaces represent the higher ESP values, dominated by nuclear effects, and the blue-colored surface regions represent lower values of the ESP, dominated by electron charges. The yellow and blue spheres in FIG. 8 indicate the local maxima and minima on the surface, respectively. Similar ESP surfaces are obtained for SM1, SM2, and SM4; in contrast, the ESP surface of SM3 is dominated by positive values. Overall, the local minima are found around the nitrogen and oxygen sites, while the local maxima are mainly located around hydrogen atoms.

A more quantitative analysis of the ESP is obtained by looking at the GIPF values. SM4 has the smallest Vₘ and SA, followed by SM1, and only minor differences are observed between SM2 and SM3. Consistent with FIG. 8, SM3 is dominated by a positive potential, as observed by the predicted values of Vₘₐₓ and Vₘᵢₙ. The Molecular Polarity Index (MPI) is a good quantitative measure of molecular polarity related to non-uniform charge distributions, in which a higher MPI value indicates greater molecular polarity, which can significantly affect the molecular behavior in the condensed phase. Considering the evaluated molecules, SM3 presents the largest MPI, followed by SM1, SM2, and SM4.

As mentioned before, CDFT properties can be used to infer the reactivity of SM1, SM2, SM3, and SM4. SM3 presented the largest global χ and ω, related to a stronger electron-molecule interaction and a greater tendency to accept electrons. Considering the polarizability of the molecules, SM4 is found to have the largest η, followed by SM3, SM1, and SM3.

Finally, considering the bond strength of the N—C sites (sites 4 and 6 of SM1-SM3 and sites 4 and 8 of SM4), the optimized conformer bond lengths correspond to 1.413, 1.410, 1.467, and 1.419 Å for SM1, SM2, SM3, and SM4, respectively. This suggests that the SM3 N—C bond is slightly weaker than those of SM1, SM2, and SM3. We explicitly evaluate the conformer energy as a function of the distance between the N and C atoms. Similar profiles are observed for SM1, SM2, and SM4; however, a broader potential profile is obtained for SM3, suggesting a relatively weaker C—N bond.

Synthesis of Value-Added Small Molecules from TBIs

The reactivity of TBI products obtained by imidazolysis was also considered. The concept of TBIs or “activating” carbonyl bisimidazole compounds can be traced back to Gerngross' pioneering work in 1913 with the preparation of N-benzoylimidazole. Later, significant progress was obtained with the preparation of other acetylimidazoles and most importantly, through the invention of carbonyldiimidazole (CDI), and a solid foundation about the chemistry and reactivity of TBIs had been established by Staab.⁶⁷ According to his findings, the acyl-substituted pyrrole-like N(1) in these heterocyclic azolides possesses a lone pair of electrons that can undergo delocalization within the azole-π-system along with the pyridine-like N(3). This delocalization results in a partial positive charge on either N atom and intensifies the electron-withdrawing influence on the C atom of the C═O group. Consequently, the acyl moiety within the azolides becomes highly susceptible to nucleophilic attack.³¹ The reactive nature of TBIs obtained from PET have been analyzed via there different methods of reaction as depicted in Scheme 4.

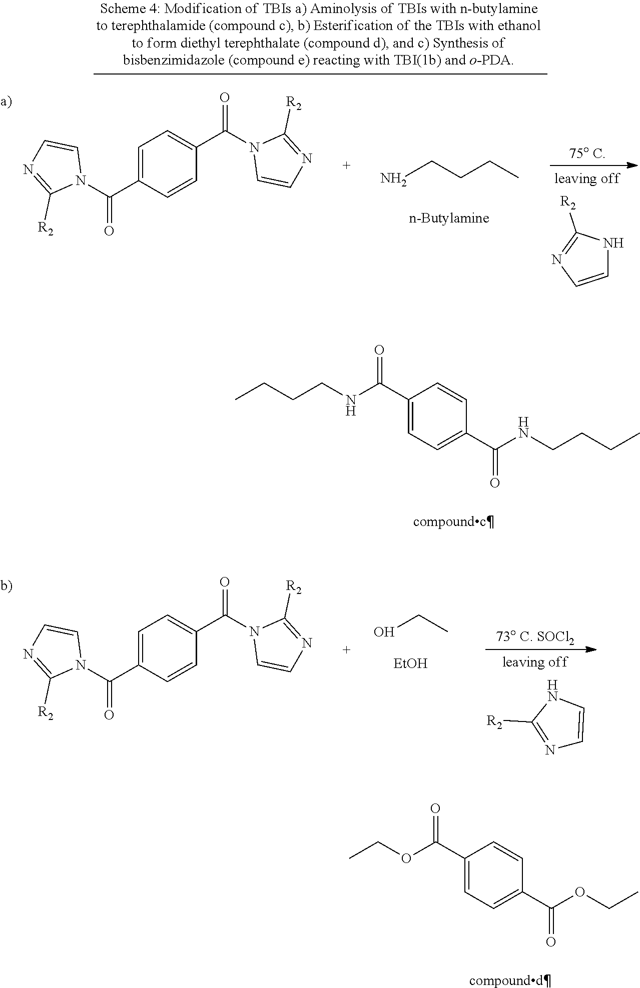

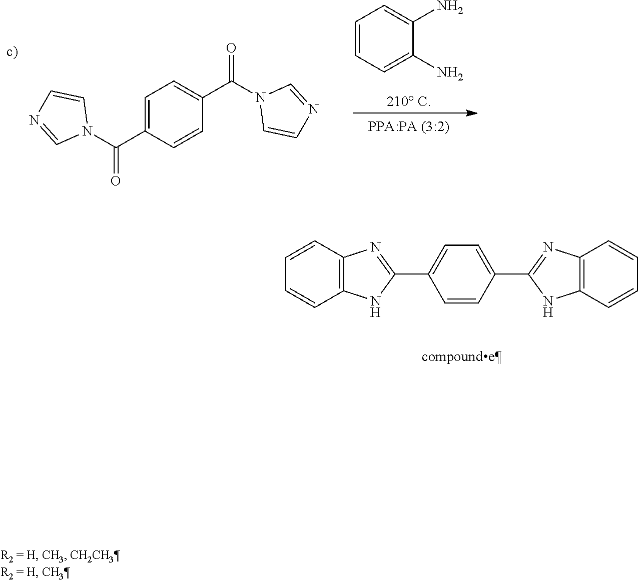 [SMILES: O=C(c1ccc(C(=O)n2ccnc2)cc1)n1ccnc1 ; Nc1ccccc1N ; c1ccc2[nH]c(-c3ccc(-c4nc5ccccc5[nH]4)cc3)nc2c1]

### Aminolysis of Carbonyl Bisimidazoles

The transacylation activity of imidazolides or carbonyl diimidazolides has been extensively investigated by previous researchers.^{36, 40, 68} Following this, our upcycling study aimed to explore the reactivity of TBIs in aminolysis reactions with excess n-butylamine under reflux. It was observed that all three TBIs (1b, 2b, and 3b) exhibited sufficient reactivity to form the symmetric 2° terephthalamide (compound c) in Scheme 4a, with moderate to high yield, as the imidazoles were displaced in favor of amide formation with amines. While TBIs incorporated with alkyl groups (2b and 3b) could be more potent than the simple TBI (1b), they might exhibit less susceptibility towards amidation than 1b, leading to lower yields. Amidation product (c) was obtained from 1b, 2b, and 3b with 86.5%, 74%, and 72.8% yields, respectively. The integration of the requisite ¹H NMR signals indicates that the N—H proton might overlap with the benzene ring around δ=8 ppm, confirmed by integrating all signal peaks. All the —CH₂— ¹H NMR signals appear in the range of δ=1.25-2.8 ppm with the terminal —CH₃ group at δ=1 ppm. The ¹H and ¹³C NMR spectra of the same product (c) prepared from 2b and 3b was also obtained.

### Esterification of Carbonyl Bisimidazoles

The reversion of TBIs to ester products was also considered. It was found that simply adding TBIs to excess alcohol (e.g., MeOH, EtOH, n-BuOH) at room temperature was not sufficient to release the imidazole and form the corresponding esters unless an acid catalyst was used. Prior reports suggested that the formation of esters from TBIs and alcohols was instantaneous, but such reactions were not observed in this work.⁶⁹ Instead, esterification was promoted using H₂SO₄ or SOCl₂ as catalysts in EtOH or MeOH (Scheme 4b). For example, TBI 1b (0.78 g, 2.92 mmol) was added in a round bottom flask with excess EtOH (12 mL) with SOCl₂ (2.0 mL), and the mixture became fully soluble when heated at reflux. After the reaction, the crude mixture was washed with diethyl ether (Et₂O) and aq. NaHCO₃ (pH=9). Finally, the product (compound d) was obtained via drying the organic layer (67% yield). TBI products 1b and 2b were successfully transformed into the corresponding esters reacting with EtOH and MeOH, respectively.

The ¹H NMR spectrum for the product of TBI (1b) with EtOH in CDCl₃ clearly supports the diethylterephthalate (DET) formation as the product. Similarly, TBI (1b) produces DMT when reacted with MeOH (79% yield). TBI (2b) exhibited equal reactivity to form esters (DET and DMT) with alcohols in the presence of SOCl₂.

### Preparation of 2,2′-(1,4-Phenylene)bis[1H-benzimidazole] Using TBI

Benzimidazoles comprise a significant class of imidazole derivatives, having been found in their practical applications in pharmacology⁷⁰, dye-sensitized solar cells⁷¹, and thermally stable poly(benzimidazole) materials.⁷² Previously, Fukushima et al. reported obtaining similar compound through organocatalytic depolymerization of PET with o-PDA (8 eq.) and TBD catalyst at 190° C. after 72 h with 62% yield.⁷³ Herein, TBIs are shown to be able to form 2,2′-bisbenzimidazole (compound e) with o-PDA (2.1 eq.) via solvent assisted method after 6 h with 86% yield. To date, several synthetic approaches have been developed for this compound. Among them, the most effective method involves the acid-catalyzed condensation of o-PDA with TA, terephthalaldehyde, or terephthalonitrile, leading to ring closure. The current results validate the use of TBI (1b) as a suitable reagent for this purpose.

As shown in Scheme 4c, 13.87 mmol of o-PDA, 6.61 mmol of 1b, and a 60 mL mixture of PPA and H₃PO₄ (3:2 ratio) were taken in a 250 mL round-bottom flask under a N₂ atmosphere. The initial reaction temperature was set to 70° C. and was gradually increased to 210° C. over 6 h. After this time, the reaction was allowed to cool and was poured into a chilled solution of 15 wt % aq. NaOH and a precipitate immediately formed. Additional aq. NaOH was added dropwise to achieve a solution of pH=7, whereby a pale green solid powder was obtained and collected via vacuum filtration. The product was purified by recrystallization from abs. EtOH yielding an off-white powder, which was dried at 80° C. under vacuum. The ¹H NMR spectrum of this compound in DMSO-d₆ was obtained. Distinguished N—H protons at δ 13 ppm and bisbenzimidazole protons at δ 7 to 8 ppm confirm the formation of the product. In addition to the purity obtained, the FTIR spectrum revealed the absence of C═O stretching band between 1780 and 1650 cm⁻¹, and N—H stretching was found around 3059 cm⁻¹, confirming the product formation.

### CONCLUSION

This example has shown that imidazole and related compounds are effective agents for depolymerizing PET without the need for a cosolvent, forming symmetric TBI compounds. Imidazolysis adds a tool for plastic waste upcycling, and the resulting TBI compounds can be used as versatile intermediates to convert to other small molecules. These results confirm that TBIs can be converted valuable terephthalates and terephthalamides, highlighting the production of TA, DMT, and bisbenzimidazole. Notably, the methodology employed here excludes the extreme reaction conditions to recover TA and DMT, typically compared to the direct hydrolysis and methanolysis of PET, offering a moderate and potentially more sustainable alternative for material recovery from polyester waste.

Therefore, the resultant TBIs have terephthaloyl moieties which demonstrate advantageous electrophilic properties, and present opportunities for more types of reactions/products.

### REFERENCES FOR EXAMPLE 3

(1) Kumartasli, S.; Avinc, O. Important step in sustainability: polyethylene terephthalate recycling and the recent developments. Sustainability in the Textile and Apparel Industries: Sourcing Synthetic and Novel Alternative Raw Materials 2020, 1-19.
(2) Zhou, D.; Chen, J.; Wu, J.; Yang, J.; Wang, H. Biodegradation and catalytic-chemical degradation strategies to mitigate microplastic pollution. Sustainable Materials and Technologies 2021, 28, e00251.
(3) Peng, Y.; Yang, J.; Deng, C.; Deng, J.; Shen, L.; Fu, Y. Acetolysis of waste polyethylene terephthalate for upcycling and life-cycle assessment study. Nature Communications 2023, 14 (1), 3249.
(4) Tournier, V.; Topham, C.; Gilles, A.; David, B.; Folgoas, C.; Moya-Leclair, E.; Kamionka, E.; Desrousseaux, M.-L.; Texier, H.; Gavalda, S. An engineered PET depolymerase to break down and recycle plastic bottles. Nature 2020, 580 (7802), 216-219.
(5) Tan, J. P.; Tan, J.; Park, N.; Xu, K.; Chan, E. D.; Yang, C.; Piunova, V. A.; Ji, Z.; Lim, A.; Shao, J. Upcycling poly (ethylene terephthalate) refuse to advanced therapeutics for the treatment of nosocomial and mycobacterial infections. Macromolecules 2019, 52 (20), 7878-7885.
(6) Jehanno, C.; Alty, J. W.; Roosen, M.; De Meester, S.; Dove, A. P.; Chen, E. Y.-X.; Leibfarth, F. A.; Sardon, H. Critical advances and future opportunities in upcycling commodity polymers. Nature 2022, 603 (7903), 803-814.
(7) Smith, R. L.; Takkellapati, S.; Riegerix, R. C. Recycling of plastics in the United States: plastic material flows and polyethylene terephthalate (PET) recycling processes. ACS sustainable chemistry & engineering 2022, 10 (6), 2084-2096.
(8) Demarteau, J.; Olazabal, I.; Jehanno, C.; Sardon, H. Aminolytic upcycling of poly (ethylene terephthalate) wastes using a thermally-stable organocatalyst. Polymer Chemistry 2020, 11 (30), 4875-4882.
(9) de Dios Caputto, M. D.; Navarro, R.; Valentin, J. L.; Marcos-Fernández, Á. Chemical upcycling of poly (ethylene terephthalate) waste: Moving to a circular model. Journal of Polymer Science 2022, 60 (24), 3269-3283.
(10) Kang, M. J.; Yu, H. J.; Jegal, J.; Kim, H. S.; Cha, H. G. Depolymerization of PET into terephthalic acid in neutral media catalyzed by the ZSM-5 acidic catalyst. Chemical Engineering Journal 2020, 398, 125655.
(11) Wang, Y.; Zhang, Y.; Song, H.; Wang, Y.; Deng, T.; Hou, X. Zinc-catalyzed ester bond cleavage: Chemical degradation of polyethylene terephthalate. Journal of Cleaner Production 2019,208, 1469-1475.
(12) Kurokawa, H.; Ohshima, M.-a.; Sugiyama, K.; Miura, H. Methanolysis of polyethylene terephthalate (PET) in the presence of aluminium tiisopropoxide catalyst to form dimethyl terephthalate and ethylene glycol. Polymer Degradation and Stability 2003, 79 (3), 529-533.
(13) Genta, M.; Iwaya, T.; Sasaki, M.; Goto, M.; Hirose, T. Depolymerization mechanism of poly (ethylene terephthalate) in supercritical methanol. Industrial & engineering chemistry research 2005, 44 (11), 3894-3900.
(14) Delle Chiaie, K. R.; McMahon, F. R.; Williams, E. J.; Price, M. J.; Dove, A. P. Dual-catalytic depolymerization of polyethylene terephthalate (PET). Polymer Chemistry 2020, 11 (8), 1450-1453.
(15) Fang, P.; Liu, B.; Xu, J.; Zhou, Q.; Zhang, S.; Ma, J. High-efficiency glycolysis of poly (ethylene terephthalate) by sandwich-structure polyoxometalate catalyst with two active sites. Polymer Degradation and Stability 2018, 156, 22-31.
(16) Fukushima, K.; Lecuyer, J. M.; Wei, D. S.; Horn, H. W.; Jones, G. O.; Al-Megren, H. A.; Alabdulrahman, A. M.; Alsewailem, F. D.; McNeil, M. A.; Rice, J. E. Advanced chemical recycling of poly (ethylene terephthalate) through organocatalytic aminolysis. Polymer Chemistry 2013, 4 (5), 1610-1616.
(17) Mittal, A.; Soni, R. K.; Dutt, K.; Singh, S. Scanning electron microscopic study of hazardous waste flakes of polyethylene terephthalate (PET) by aminolysis and ammonolysis. J Hazard Mater 2010, 178 (1-3), 390-396.
(18) George, N.; Kurian, T. Recent developments in the chemical recycling of postconsumer poly (ethylene terephthalate) waste. Industrial & Engineering Chemistry Research 2014, 53 (37), 14185-14198.
(19) Payne, J. M.; Kamran, M.; Davidson, M. G.; Jones, M. D. Versatile Chemical Recycling Strategies: Value-Added Chemicals from Polyester and Polycarbonate Waste. ChemSusChem 2022, 15 (8), e202200255.
(20) Natarajan, J.; Madras, G.; Chatterjee, K. Poly (ester amide) s from Poly (ethylene terephthalate) Waste for Enhancing Bone Regeneration and Controlled Release. ACS Applied Materials & Interfaces 2017, 9 (34), 28281-28297.
(21) Kulkarni, A.; Quintens, G.; Pitet, L. M. Trends in polyester upcycling for diversifying a problematic waste stream. Macromolecules 2023, 56 (5), 1747-1758.
(22) Demarteau, J.; O'Harra, K. E.; Bara, J. E.; Sardon, H. Valorization of Plastic Wastes for the Synthesis of Imidazolium-Based Self-Supported Elastomeric Ionenes. ChemSusChem 2020, 13 (12), 3122-3126.
(23) O'Harra, K.; Sadaba, N.; Irigoyen, M.; Ruipérez, F.; Aguirresarobe, R.; Sardon, H.; Bara, J. Nearly perfect 3D structures obtained by assembly of printed parts of polyamide ionene self-healing elastomer. ACS Applied Polymer Materials 2020, 2 (11), 4352-4359.
(24) Zhang, L.; Peng, X. M.; Damu, G. L.; Geng, R. X.; Zhou, C. H. Comprehensive review in current developments of imidazole-based medicinal chemistry. Medicinal research reviews 2014, 34 (2), 340-437.
(25) O'Harra, K. E.; Bara, J. E. Toward controlled functional sequencing and hierarchical structuring in imidazolium ionenes. Polymer International 2021, 70 (7), 944-950.
(26) Gujjarappa, R.; Kabi, A. K.; Sravani, S.; Garg, A.; Vodnala, N.; Tyagi, U.; Kaldhi, D.; Velayutham, R.; Singh, V.; Gupta, S. Overview on biological activities of imidazole derivatives. In Nanostructured Biomaterials: Basic Structures and Applications, Springer, 2022; pp 135-227.
(27) Vinggaard, A. M.; Hass, U.; Dalgaard, M.; Andersen, H. R.; Bonefeld-Jergensen, E.; Christiansen, S.; Laier, P.; Poulsen, M. E. Prochloraz: an imidazole fungicide with multiple mechanisms of action. International journal of andrology 2006, 29 (1), 186-192.
(28) Bara, J. E. Versatile and scalable method for producing N-functionalized imidazoles. Industrial & engineering chemistry research 2011, 50 (24), 13614-13619.
(29) Ananthu, S.; Aneeja, T.; Anilkumar, G. N-Arylation of Imidazoles: An Overview. ChemistrySelect 2021, 6 (37), 9794-9805.
(30) Liu, L.; Huang, Y.; Riduan, S. N.; Gao, S.; Yang, Y.; Fan, W.; Zhang, Y. Main-chain imidazolium oligomer material as a selective biomimetic antimicrobial agent. Biomaterials 2012, 33 (33), 8625-8631.
(31) Staab, H. A.; Bauer, H.; Schneider, K. M. Azolides in organic synthesis and biochemistry; Wiley-VCH, 1998.
(32) Olazabal, I.; Luna, E.; De Meester, S.; Jehanno, C.; Sardon, H. Upcycling of BPA-PC into trimethylene carbonate by solvent assisted organocatalysed depolymerisation. Polymer Chemistry 2023, 14 (19), 2299-2307.
(33) Boyer, J. The acetylation of imidazole. Journal of the American Chemical Society 1952, 74 (24), 6274-6275.
(34) Schmeer, G.; Six, C.; Steinkirchner, J. Investigations on substituent and solvent effects of solvolysis reactions. VIII. The influence of water and nonaqueous solvents on the imidazolysis of 4-nitrophenyl acetate. Journal of solution chemistry 1999, 28, 211-222.
(35) Rivetti, F.; Tonellato, U. Kinetics of ester imidazolysis in benzene. Journal of the Chemical Society, Perkin Transactions 2 1977, (9), 1176-1179, 10.1039/P29770001176.
(36) Mino, T.; Sakamoto, S.; Hamachi, I. Recent applications of N-acyl imidazole chemistry in chemical biology. Bioscience, Biotechnology, and Biochemistry 2021, 85 (1), 53-60.
(37) Quan, H.; Wang, L.; Wang, Z.; Mei, X.; Ning, J.; She, D. Application of N-Acylimidazoles in the Claisen Condensation Reaction. ChemistrySelect 2020, 5 (24), 7222-7226.
(38) Lanzillotto, M.; Konnert, L.; Lamaty, F.; Martinez, J.; Colacino, E. Mechanochemical 1, 1′-carbonyldiimidazole-mediated synthesis of carbamates. ACS Sustainable Chemistry & Engineering 2015, 3 (11), 2882-2889.
(39) Grzyb, J. A.; Shen, M.; Yoshina-Ishii, C.; Chi, W.; Brown, R. S.; Batey, R. A. Carbamoylimidazolium and thiocarbamoylimidazolium salts: novel reagents for the synthesis of ureas, thioureas, carbamates, thiocarbamates and amides. Tetrahedron 2005, 61 (30), 7153-7175.
(40) Staab, v. H. New methods of preparative organic chemistry IV. Syntheses using heterocyclic amides (azolides). Angewandte Chemie International Edition in English 1962, 1 (7), 351-367.
(41) Evjen, S.; Fiksdahl, A. Syntheses of polyalkylated imidazoles. Synth. Commun. 2017, 47 (15), 1392-1399.
(42) O'Boyle, N. M.; Banck, M.; James, C. A.; Morley, C.; Vandermeersch, T.; Hutchison, G. R. Open Babel: An open chemical toolbox. Journal of cheminformatics 2011, 3 (1), 1-14.
(43) Lee, C.; Yang, W.; Parr, R. G. Development of the Colle-Salvetti correlation-energy formula into a functional of the electron density. Physical review B 1988, 37(2), 785.
(44) Becke, A. D. A new mixing of Hartree-Fock and local density-functional theories. The Journal of chemical physics 1993, 98 (2), 1372-1377.
(45) Petersson, a.; Bennett, A.; Tensfeldt, T. G.; Al-Laham, M. A.; Shirley, W. A.; Mantzaris, J. A complete basis set model chemistry. I. The total energies of closed-shell atoms and hydrides of the first-row elements. The Journal of chemical physics 1988, 89 (4), 2193-2218.
(46) Petersson, G.; Al-Laham, M. A. A complete basis set model chemistry. II. Open-shell systems and the total energies of the first-row atoms. The Journal of chemical physics 1991, 94 (9), 6081-6090.
(47) Parr, R. G.; Pearson, R. G. Absolute hardness: companion parameter to absolute electronegativity. Journal of the American chemical society 1983, 105 (26), 7512-7516.
(48) Yang, W.; Parr, R. G. Hardness, softness, and the fukui function in the electronic theory of metals and catalysis. Proceedings of the National Academy of Sciences 1985, 82 (20), 6723-6726.
(49) Parr, R. G.; Szentpály, L. v.; Liu, S. Electrophilicity index. Journal of the American Chemical Society 1999, 121 (9), 1922-1924.
(50) Lu, T.; Chen, Q. Realization of Conceptual Density Functional Theory and Information-Theoretic Approach in Multiwfn Program. Conceptual Density Functional Theory: Towards a New Chemical Reactivity Theory 2022, 2, 631-647.
(51) Clark, T.; Chandrasekhar, J.; Spitznagel, G. W.; Schleyer, P. V. R. Efficient diffuse function-augmented basis sets for anion calculations. III. The 3-21+ G basis set for first-row elements, Li—F. Journal of Computational Chemistry 1983, 4 (3), 294-301.
(52) Grimme, S.; Antony, J.; Ehrlich, S.; Krieg, H. A consistent and accurate ab initio parametrization of density functional dispersion correction (DFT-D) for the 94 elements H—Pu. The Journal of chemical physics 2010, 132 (15).
(53) Lu, T.; Chen, F. Multiwfn: A multifunctional wavefunction analyzer. Journal of computational chemistry 2012, 33 (5), 580-592.
(54) Zhang, J.; Lu, T. Efficient evaluation of electrostatic potential with computerized optimized code. Physical Chemistry Chemical Physics 2021, 23 (36), 20323-20328.
(55) Murray, J. S.; Brinck, T.; Lane, P.; Paulsen, K.; Politzer, P. Statistically-based interaction indices derived from molecular surface electrostatic potentials: a general interaction properties function (GIPF). Journal of Molecular Structure: THEOCHEM 1994, 307, 55-64.
(56) Lu, T.; Chen, F. Quantitative analysis of molecular surface based on improved Marching Tetrahedra algorithm. Journal of Molecular Graphics and Modelling 2012, 38, 314-323.
(57) Bader, R. F.; Carroll, M. T.; Cheeseman, J. R.; Chang, C. Properties of atoms in molecules: atomic volumes. Journal of the American Chemical Society 1987, 109 (26), 7968-7979.
(58) Lenarcik, B.; Ojczenasz, P. The influence of the size and position of the alkyl groups in alkylimidazole molecules on their acid-base properties. Journal of heterocyclic chemistry 2002, 39 (2), 287-290.
(59) Brown, R.; Huguet, J. Synthesis and physical studies of pyridine and imidazole containing tridentate metal binding ligands. Canadian Journal of Chemistry 1980, 58 (9), 889-901.
(60) Perrin, D. D.; Dempsey, B.; Serjeant, E. P. pKa prediction for organic acids and bases; Springer, 1981.
(61) Razzaq, T.; Kappe, C. O. On the energy efficiency of microwave-assisted organic reactions. ChemSusChem: Chemistry & Sustainability Energy & Materials 2006, 1 (1-2), 123-132.
(62) Ogiwara, Y.; Nomura, K. Chemical Upcycling of PET into a Morpholine Amide as a Versatile Synthetic Building Block. ACS Organic & Inorganic Au 2023, 3 (6), 377-383.
(63) Schofield, K.; Grimmett, M. R.; Keene, B. R. T. Heteroaromatic nitrogen compounds: the azoles; CUP Archive, 1976.
(64) Socrates, G. Infrared and Raman characteristic group frequencies: tables and charts; John Wiley & Sons, 2004.
(65) Martinez, J. M. L.; Denis, M. F. L.; Dall'Orto, V. C.; Buldain, G. Y. Synthesis, FTIR, solid-state NMR and SEM studies of novel polyampholytes or polyelectrolytes obtained from EGDE, MAA and imidazoles. European polymer journal 2008, 44 (2), 392-407.
(66) Kalsi, P. Spectroscopy of organic compounds; New age international, 2007.
(67) Wieland, T.; Schneider, G. N-acylimidazoles as acyl derivatives of high energy. Ann. Chem. Justus Liebigs 1953, 580, 159.
(68) Bansagi, J.; Wilson-Konderka, C.; Debrauwer, V.; Narayanan, P.; Batey, R. A. N-Alkyl Carbamoylimidazoles as Isocyanate Equivalents: Exploration of the Reaction Scope for the Synthesis of Ureas, Hydantoins, Carbamates, Thiocarbamates, and Oxazolidinones. The Journal of Organic Chemistry 2022, 87(17), 11329-11349.
(69) Staab, H. A.; Bauer, H.; Schneider, K. M. Azolides in organic synthesis and biochemistry. 1998.
(70) Valdez, J.; Cedillo, R.; Hernández-Campos, A.; Yepez, L.; Hernández-Luis, F.; Navarrete-Vazquez, G.; Tapia, A.; Cortés, R.; Hernández, M.; Castillo, R. Synthesis and antiparasitic activity of 1H-benzimidazole derivatives. Bioorganic & Medicinal Chemistry Letters 2002, 12 (16), 2221-2224.
(71) Saltan, G. M.; Dinçalp, H.; Kiran, M.; Zafer, C.; Erbaş, S. Ç. Novel organic dyes based on phenyl-substituted benzimidazole for dye sensitized solar cells. Materials Chemistry and Physics 2015, 163, 387-393.
(72) Shang, Z.; Yang, L.; Chang, G. Soluble N-substituted poly (benzimidazole imide) s via C—N coupling reaction. Polymer International 2016, 65 (3), 332-338.
(73) Fukushima, K.; Jones, G. O.; Horn, H. W.; Rice, J. E.; Kato, T.; Hedrick, J. L. Formation of bis-benzimidazole and bis-benzoxazole through organocatalytic depolymerization of poly (ethylene terephthalate) and its mechanism. Polymer Chemistry 2020, 11 (30), 4904-4913.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

What is claimed is:

1. A process for depolymerizing a polyester comprising treating the polyester with an optionally substituted imidazole to form one or more products comprising one or more optionally substituted 1-acyl imidazole moieties.

2. The process of claim 1, wherein the polyester was formed by condensation of a dicarboxylic acid and a polyol.

3. The process of claim 1, wherein the polyester is selected from poly(ethylene terephthalate) (PET), poly(trimethylene terephthalate) (PTT), poly(butylene terephthalate) (PBT), poly(ethylene isophthalate), poly(octamethylene terephthalate), poly(decamethylene terephthalate), poly(pentamethylene isophthalate), poly(butylene isophthalate), poly(hexamethylene isophthalate), poly(hexamethylene adipate), poly(pentamethylene adipate), poly(pentamethylene sebacate), poly(hexamethylene sebacate), poly(1,4-cyclohexylene terephthalate), poly(1,4-cyclohexylene sebacate), poly(ethylene terephthalate-co-sebacate), and poly(ethylene-co-tetramethylene terephthalate).

4. The process of claim 1, wherein the one or more products comprise one or more 1-acyl imidazole derivatives of terephthalic acid, isophthalic acid, 2,6-naphthalene dicarboxylic acid, 4,4′-diphenylether dicarboxylic acid, hexahydrophthalic acid, 2,7-naphthalenedicarboxylic acid, phthalic acid, 4,4′-methylenebis(benzoic acid), oxalic acid, malonic acid, succinic acid, methyl succinic acid, glutaric acid, adipic acid, 3-methyladipic acid, pimelic acid, suberic acid, azelaic acid, sebacic acid, 1,11-undecanedicarboxylic acid, 1,10-decanedicarboxylic acid, hexadecanedioic acid, docosanedioic acid, tetracosanedioic acid, 1,4-cyclohexanedicarboxylic acid, 1,3-cyclohexanedicarboxylic acid, 1,2-cyclohexanediacetic acid, fumaric acid, maleic acid, or combinations thereof.

5. The process of claim 1, wherein the polyester was formed by condensation of a hydroxy-substituted acid or ester or derivative thereof, such as a lactone.

6. The process of claim 1, wherein the polyester is selected from polyglycolic acid (PGA), polylactic acid (PLA), polycaprolactone (PCL), polyhydroxybutyrate (PHB), or a polyhydroxyalkanoate (PHA).

7. The process of claim 1, wherein the one or more products comprise one or more 1-acyl imidazole derivatives of glycolic acid, lactic acid, hydroxycaproic acid, hydroxybutyric acid, or a hydroxyalkanoic acid, or combinations thereof.

8. The process of claim 1, wherein the polyester comprises polyethylene terephthalate (PET).

9. The process of claim 1, wherein the optionally substituted imidazole comprises a compound of Formula I 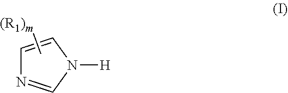
wherein
m is 0, 1, 2, or 3; and
R₁ is independently selected at each occurrence from hydrogen, halo, cyano, azido, nitro, silyl, hydroxy, amino, aldehyde, carboxylic acid, ester, C₁-C₆ alkyl, C₁-C₆ haloalkyl, C₂-C₆ alkenyl, C₂-C₆ alkynyl, C₁-C₆ alkoxy, C₃-C₆ cycloalkyl, 3- to 8-membered monocyclic or bicyclic heterocycle, 6- to 10-membered monocyclic or bicyclic aryl, 5- to 10-membered monocyclic or bicyclic heteroaryl, sulfo-oxo, sulfonyl, sulfone, sulfoxide, sulfonylamino, and thiol.

10. The process of claim 1, wherein the optionally substituted imidazole is selected from imidazole, 2-ethylimidazole, 2,4,5-trimethylimidazole, 2-isopropylimidazole, and 4-methylimidazole.

11. The process of claim 1, wherein the optionally substituted imidazole is present in an amount of about 2 molar equivalents to about 10 molar equivalents relative to the polyester.

12. The process of claim 1, wherein the process is performed at a temperature from about 100 degrees Celsius to about 200 degrees Celsius.

13. A compound comprising one or more optionally substituted 1-acyl imidazole moieties formed by the process of claim 1.

14. A process for depolymerizing a polyurethane comprising treating the polyurethane with an optionally substituted imidazole to form one or more products having one or more optionally substituted 1-carboxamide imidazole moieties.

15. The process of claim 14, wherein the polyurethane was formed by condensation of one or more diisocyanates, one or more polyols, optionally one or more chain extenders, and optionally one or more crosslinking agents.

16. The process of claim 15, wherein the one or more diisocyanates are selected from an aliphatic diisocyanate, an aromatic diisocyanate, or combinations thereof.

17. The process of claim 15, wherein the one or more diisocyanates are selected from 4,4′-dicyclohexylmethane diisocyanate, isophorone diisocyanate, 4,4′-diphenylmethane diisocyanate, 2,4-toluene diisocyanate, 2,6-toluene diisocyanate, hexamethylene diisocyanate, or combinations thereof.

18. The process of claim 15, wherein the one or more polyols are selected from a polyether polyol, a polycarbonate polyol, a polyester polyol, a silicone polyol, or combinations thereof.

19. The process of claim 15, wherein the one or more polyols are selected from polyethylene glycol, polypropylene glycol, polytetramethyleneglycol, poly(1,6-hexamethylene carbonate)diol, poly(decamethylene carbonate)diol, oligocarbonate diol, poly(hexamethylene-pentamethylene carbonate)diol, polylactide, polycaprolactone, polyethylene glycol adipate, polydimethylsiloxane, polyaryl siloxane, and polyalkyl siloxane.

20. The process of claim 14, wherein one or more products comprise one or more 1-carboxamide imidazole derivatives of 4,4′-dicyclohexylmethane diisocyanate, isophorone diisocyanate, 1,4-cyclohexylmethane diisocyanate, 4,4′-diphenylmethane diisocyanate, 2,4-toluene diisocyanate, 2,6-toluene diisocyanate, hexamethylene diisocyanate, or combinations thereof.

21. The process of claim 14, wherein the optionally substituted imidazole comprises a compound of Formula I 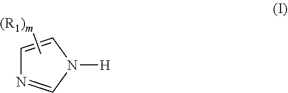
wherein
m is 0, 1, 2, or 3; and
R₁ is independently selected at each occurrence from hydrogen, halo, cyano, azido, nitro, silyl, hydroxy, amino, aldehyde, carboxylic acid, ester, C₁-C₆ alkyl, C₁-C₆ haloalkyl, C₂-C₆ alkenyl, C₂-C₆ alkynyl, C₁-C₆ alkoxy, C₃-C₆ cycloalkyl, 3- to 8-membered monocyclic or bicyclic heterocycle, 6- to 10-membered monocyclic or bicyclic aryl, 5- to 10-membered monocyclic or bicyclic heteroaryl, sulfo-oxo, sulfonyl, sulfone, sulfoxide, sulfonylamino, and thiol.

22. The process of claim 14, wherein the optionally substituted imidazole is selected from imidazole, 2-ethylimidazole, 2,4,5-trimethylimidazole, 2-isopropylimidazole, and 4-methylimidazole.

23. A compound comprising one or more optionally substituted 1-carboxamide imidazole moieties formed by the process of claim 14.
